SEQUENCE LISTING (38 sequences)

SEQ ID NO: 1 - DNA, 1212 bp, Candida macedoniensis
  CDS (1)..(1212)
atgtcgtacatgaactttgaccctaagccattgggagacaccaatatcttcaagccaatcaagatcggtaacaatgagctaaaacacagagtagtcatgccagcattgactagaatgagagccattgcaccaggaaacatcccaaacactgaatgggccgaggaatactacagacaacgttctcaataccctggtacccttattatcacggaaggtactttcccttctgcgcaatcaggtggttacccaaatgtgccaggtatctggtccaaagagcaattggctgaatggaaaaagatcttcaatgcaatccatgagaacaaatcgttcgtgtgggtgcaattgtgggttctaggtagacaagcatggccagaagtgttgaagaaggaaggtttgcgttacgatagtgctaccgatgacttgtacatgggtgaagaagaaaaagagcgtgccttaaaggctaacaacccacagcacggtatcaccaaggaagaaatcaagcagtacatcaaggagtacgtggatgctgccaagaaagccatcgatgcaggtgcagacggtgtgcaaatccattctgccaacggttacttgttgaaccagtttttggaccctatttctaacaacagaaccgacgagtacggtggatcgatcgagaaccgtgcgagattcactttggaagtggtcgatgccgttgtcgatgcagttggtgccgaaagaacctccatcagattctctccatacggtacttttggtaccatgtccggtggtgagaaccctggcatcgttgctcaatatgcatacgtcattggtgagttggaaaagagagctagagctggcaagagattggcgttcatcgatttggtcgagcctcgtgtgaccgacccattcctaccagaattcgagaagtggttcaaggaaggtaccaacgaattcatctactctatctggaagggtccagttctcagagttggtaactatgctttggacccagatcaagccactctcgactctaagaagcctaacactttgatcggttacggtagatccttcatcgccaacccagacttggtgtaccgtttggaaaagggtttgccattgaacaagtatgatagaaacaccttttacacattcactaaggaaggttacaccgattacccaagctacgaagaatccgtcgcaaagggttacaagaaagaggaaaagaagtactaa

SEQ ID NO: 2 - protein, 403 aa, Candida macedoniensis
MSYMNFDPKPLGDTNIFKPIKIGNNELKHRVVMPALTRMRAIAPGNIPNTEWAEEYYRQRSQYPGTLIITEGTFPSAQSGGYPNVPGIWSKEQLAEWKKIFNAIHENKSFVWVQLWVLGRQAWPEVLKKEGLRYDSATDDLYMGEEEKERALKANNPQHGITKEEIKQYIKEYVDAAKKAIDAGADGVQIHSANGYLLNQFLDPISNNRTDEYGGSIENRARFTLEVVDAVVDAVGAERTSIRFSPYGTFGTMSGGENPGIVAQYAYVIGELEKRARAGKRLAFIDLVEPRVTDPFLPEFEKWFKEGTNEFIYSIWKGPVLRVGNYALDPDQATLDSKKPNTLIGYGRSFIANPDLVYRLEKGLPLNKYDRNTFYTFTKEGYTDYPSYEESVAKGYKKEEKKY

SEQ ID NO: 3 - DNA, 1197 bp, Kluyveromyces lactis
  CDS (1)..(1197)
atgtcgtttatgaactttgaaccaaagccattggctgatactgatatcttcaaaccaatcaagattggtaacactgaattgaagcacagggttgtcatgcctgcattgacaagaatgagagcgttgcatccaggcaacgttccaaaccctgactgggctgttgaatattacagacaacgttcccaatatccaggtactatgattatcactgaaggtgctttcccatcagctcagtcaggtggttacgataacgcaccaggtgtttggagcgaagaacaactggctcaatggagaaagatcttcaaggcaattcacgacaacaagtcttttgtttgggtacaattgtgggttctaggtagacaagcttttgctgataacttggcaagagatggattgcgttatgatagtgcttccgatgaagtgtacatgggtgaagatgaaaaggaacgtgccatcagatctaacaaccctcagcatggtatcaccaaggatgaaattaagcagtatatcagggactatgttgatgctgctaagaagtgtatcgatgctggtgcagatggtgttgaaatccattccgctaacggttatttgttgaatcaattcctagacccaatctccaacaaaagaactgatgaatacggtggatccattgagaaccgtgctagattcgtcttggaagtcgtcgatgccgttgtcgatgccgttggtgccgaaagaaccagtatcagattctcaccatacggtgtatttggtaccatgtcaggtgtttcagaccctgtcttggtggctcaattcgcctatgtacttgctgaattggaaaagagggcaaaggctggtaagagattagcatacgtcgatttagtcgaacctcgtgtcacatcgccattccaaccggaatttgaaggctggtataaaggtggtaccaatgaattcgtatactctgtttggaagggtaacgtgctaagagttggtaactacgctttggacccagatgctgccattacggactcaaagaatccaaacactttgatcggttacggtagagccttcattgccaacccagatcttgttgaacgtctcgaaaagggtttgccattgaatcaatacgatagaccctctttctacaaaatgtctgcggaagggtatatcgactacccaacatacgaggaagctgttgccaagggttacaagaaatag

SEQ ID NO: 4 - protein, 398 aa, Kluyveromyces lactis
MSFMNFEPKPLADTDIFKPIKIGNTELKHRVVMPALTRMRALHPGNVPNPDWAVEYYRQRSQYPGTMIITEGAFPSAQSGGYDNAPGVWSEEQLAQWRKIFKAIHDNKSFVWVQLWVLGRQAFADNLARDGLRYDSASDEVYMGEDEKERAIRSNNPQHGITKDEIKQYIRDYVDAAKKCIDAGADGVEIHSANGYLLNQFLDPISNKRTDEYGGSIENRARFVLEVVDAVVDAVGAERTSIRFSPYGVFGTMSGVSDPVLVAQFAYVLAELEKRAKAGKRLAYVDLVEPRVTSPFQPEFEGWYKGGTNEFVYSVWKGNVLRVGNYALDPDAAITDSKNPNTLIGYGRAFIANPDLVERLEKGLPLNQYDRPSFYKMSAEGYIDYPTYEEAVAKGYKK

SEQ ID NO: 5 - DNA, 1050 bp, Pseudomonas fluorescens
  CDS (1)..(1050)
atggcaactattttcgatccgatcaaactgggcgacctcgagctgtccaaccgcatcatcatggccccgctgactcgctgccgcgccgacgaaggccgcgtacccaacgcactgatggccgagtactacgtgcaacgtgcctccgccggcctgattctcagcgaagccacttcggtgacgccgatgggcgtcggctatccggacaccccgggcatctggtccaacgatcaggtacgcggctggaccaacatcaccaaagccgtacacgctgccggcggcaagatcgtcctgcaactttggcacgtcggccgcatctcgcacccgttgtacctgaacggcgaagcaccggtcgcgccgagcgccatccagcctaaaggccacgtcagcctggtgcgtccactggccgattacccgactccacgcgccctggaaaccgctgaaatcgccgagatcgtcgaggcctaccgcaccggtgccgagaacgccaaggccgccggtttcgacggcgtggaaatccacggcgccaacggctacctgctcgaccagttcttgcaaagcagcaccaaccagcgcaccgacaattacggcggctccctggaaaaccgtgcgcgtctgttgctggaagtgactgatgccgcgatcgacgtctggggcgccggccgtgtcggtgtgcacctggcaccgcgcgccgactcccacgacatgggcgacgacaacctcgccgagaccttcacctatgttgctcgcgagctgggcaagcgtggcatcgccttcatctgctcccgcgagaaagaaggcgccgacagcctcggcccacaactgaaagaagcctttggcggcgcgtacatcgccaacgagcgtttcaccaaggacagcgccaatgcgtggctggctgaaggcaaggctgacgctgtagcgttcggcgtgccattcattgccaacccggacctgccggcacgcctgaaagccgatgccccgctgaacgagccgcgtcctgagctgttctatggcaaaggcccggtcggctacatcgactacccgacgctgtaa

SEQ ID NO: 6 - protein, 349 aa, Pseudomonas fluorescens
MATIFDPIKLGDLELSNRIIMAPLTRCRADEGRVPNALMAEYYVQRASAGLILSEATSVTPMGVGYPDTPGIWSNDQVRGWTNITKAVHAAGGKIVLQLWHVGRISHPLYLNGEAPVAPSAIQPKGHVSLVRPLADYPTPRALETAEIAEIVEAYRTGAENAKAAGFDGVEIHGANGYLLDQFLQSSTNQRTDNYGGSLENRARLLLEVTDAAIDVWGAGRVGVHLAPRADSHDMGDDNLAETFTYVARELGKRGIAFICSREKEGADSLGPQLKEAFGGAYIANERFTKDSANAWLAEGKADAVAFGVPFIANPDLPARLKADAPLNEPRPELFYGKGPVGYIDYPTL

SEQ ID NO: 7 - DNA, 1083 bp, Pseudomonas syringae
  CDS (1)..(1083)
atgccgactcttttcgaccccttgactttgggcgacctgcaatctccaaaccgtgttctgatggcaccgctaacgcgtggccgcgcgacccgcgagcacgtgcctaccgagctgatgatcgagtattacacccagcgtgccagcgcgggcctgatcatcaccgaagccaccggcatcacccaagaaggcctaggctggccctatgcgcccggcatttggagcgatgaacaggtcgaggcctggaagccggtgacccaggccgtgcatgaggcaggcggacggatcattcttcagttgtggcatatgggccgtaccgttcattccagctttctcggcggagccaagccagtatcgtcctcggccacccgtgcgccgggacaggcgcacacctacgaaggcaagcaagactacgacgaggcgcggcctttgtcggcggatgaaatcccgcggctattgaacgattacgaacacgcagcgaaaaacgccatggccgcaggcttcgacggcgtgcagatccatgctgccaatggttacctaatcgaccagttcctgcgcgacaacagcaacgttcgcggggacgcctacgggggttcaatcgagaaccgcatccgtctattggtcgaagtcacccggcgcgtggcggagaccgtaggtgccgaaaaaacgggcgtgcggctgtcacccaacggtgattcccaaggcgtcaacgacagcaatccggagccgctgttcagcgccgcggccaaggccttggatgagatcggcatcgctcatctggagttgcgcgaaccagggtatgaaggcaccttcggcaaggccgaccggccgcccgtgcacccggtcatccgccaggcgttcagccgtacgctgattctcaactctgactacactttggaaacggctcaggctgcactagccaccggagaagcggacgcgatcaccttcggccgcccgttcctggccaaccctgacctgcctcacaggtttgccgagagactgccgctgaacaaggacgtgatggagacttggtatagccaggggcccgaaggttatgtggactaccccaccgctgaccaaaagtag

SEQ ID NO: 8 - protein, 360 aa, Pseudomonas syringae
MPTLFDPLTLGDLQSPNRVLMAPLTRGRATREHVPTELMIEYYTQRASAGLIITEATGITQEGLGWPYAPGIWSDEQVEAWKPVTQAVHEAGGRIILQLWHMGRTVHSSFLGGAKPVSSSATRAPGQAHTYEGKQDYDEARPLSADEIPRLLNDYEHAAKNAMAAGFDGVQIHAANGYLIDQFLRDNSNVRGDAYGGSIENRIRLLVEVTRRVAETVGAEKTGVRLSPNGDSQGVNDSNPEPLFSAAAKALDEIGIAHLELREPGYEGTFGKADRPPVHPVIRQAFSRTLILNSDYTLETAQAALATGEADAITFGRPFLANPDLPHRFAERLPLNKDVMETWYSQGPEGYVDYPTADQK

SEQ ID NO: 9 - DNA, 1098 bp, Escherichia coli
  CDS (1)..(1098)
atgtcatctgaaaaactgtattccccactgaaagtgggcgcgatcacggcggcaaaccgtatttttatggcaccgctgacgcgtctgcgcagtattgaaccgggtgacattcctaccccgttgatggcggaatactatcgccaacgtgccagtgccggtttgattattagtgaagccacgcaaatttctgcccaggcaaaaggatatgcaggtgcgcctggcatccatagtccggagcaaattgccgcatggaaaaaaatcaccgctggcgttcatgctgaaaatggtcatatggccgtgcagctgtggcacaccggacgcatttctcacgccagcctgcaacctggcggtcaggcaccggtagcgccttcagcacttagcgcgggaacacgtacttctctgcgcgatgaaaatggtcaggcgatccgtgttgaaacatccatgccgcgtgcgcttgaactggaagagattccaggtatcgtcaatgatttccgtcaggccattgctaacgcgcgtgaagccggttttgatctggtagagctccactctgctcacggttatttgctgcatcagttcctttctccttcttcaaaccatcgtaccgatcagtacggcggcagcgtggaaaatcgcgcacgtttggtactggaagtggtcgatgccgggattgaagaatggggtgccgatcgcattggcattcgcgtttcaccaatcggtactttccagaacacagataacggcccgaatgaagaagccgatgcactgtatctgattgaacaactgggtaaacgcggcattgcttatctgcatatgtcagaaccagattgggcggggggtgaaccgtatactgatgcgttccgcgaaaaagtacgcgcccgtttccacggtccgattatcggcgcaggtgcatacacagtagaaaaagctgaaacgctgatcggcaaagggttaattgatgcggtggcatttggtcgtgactggattgcgaacccggatctggtcgcccgcttgcagcgcaaagctgagcttaacccacagcgtgccgaaagtttctacggtggcggcgcggaaggctataccgattacccgacgttgtaa

SEQ ID NO: 10 - protein, 365 aa, Escherichia coli
MSSEKLYSPLKVGAITAANRIFMAPLTRLRSIEPGDIPTPLMAEYYRQRASAGLIISEATQISAQAKGYAGAPGIHSPEQIAAWKKITAGVHAENGHMAVQLWHTGRISHASLQPGGQAPVAPSALSAGTRTSLRDENGQAIRVETSMPRALELEEIPGIVNDFRQAIANAREAGFDLVELHSAHGYLLHQFLSPSSNHRTDQYGGSVENRARLVLEVVDAGIEEWGADRIGIRVSPIGTFQNTDNGPNEEADALYLIEQLGKRGIAYLHMSEPDWAGGEPYTDAFREKVRARFHGPIIGAGAYTVEKAETLIGKGLIDAVAFGRDWIANPDLVARLQRKAELNPQRAESFYGGGAEGYTDYPTL

SEQ ID NO: 11 - DNA, 1203 bp, Saccharomyces cerevisiae
  CDS (1)..(1203)
atgccatttgttaaggactttaagccacaagctttgggtgacaccaacttattcaaaccaatcaaaattggtaacaatgaacttctacaccgtgctgtcattcctccattgactagaatgagagcccaacatccaggtaatattccaaacagagactgggccgttgaatactacgctcaacgtgctcaaagaccaggaaccttgattatcactgaaggtacctttccctctccacaatctgggggttacgacaatgctccaggtatctggtccgaagaacaaattaaagaatggaccaagattttcaaggctattcatgagaataaatcgttcgcatgggtccaattatgggttctaggttgggctgctttcccagacacccttgctagggatggtttgcgttacgactccgcttctgacaacgtgtatatgaatgcagaacaagaagaaaaggctaagaaggctaacaacccacaacacagtataacaaaggatgaaattaagcaatacgtcaaagaatacgtccaagctgccaaaaactccattgctgctggtgccgatggtgttgaaatccacagcgctaacggttacttgttgaaccagttcttggacccacactccaataacagaaccgatgagtatggtggatccatcgaaaacagagcccgtttcaccttggaagtggttgatgcagttgtcgatgctattggccctgaaaaagtcggtttgagattgtctccatatggtgtcttcaacagtatgtctggtggtgctgaaaccggtattgttgctcaatatgcttatgtcttaggtgaactagaaagaagagctaaagctggcaagcgtttggctttcgtccatctagttgaacctcgtgtcaccaacccatttttaactgaaggtgaaggtgaatacaatggaggtagcaacaaatttgcttattctatctggaagggcccaattattagagctggtaactttgctctgcacccagaagttgtcagagaagaggtgaaggatcctagaacattgatcggttacggtagattttttatctctaatccagatttggttgatcgtttggaaaaagggttaccattaaacaaatatgacagagacactttctacaaaatgtcagctgagggatacattgactaccctacgtacgaagaagctctaaaactcggttgggacaaaaattaa

SEQ ID NO: 12 - protein, 400 aa, Saccharomyces cerevisiae
MPFVKDFKPQALGDTNLFKPIKIGNNELLHRAVIPPLTRMRAQHPGNIPNRDWAVEYYAQRAQRPGTLIITEGTFPSPQSGGYDNAPGIWSEEQIKEWTKIFKAIHENKSFAWVQLWVLGWAAFPDTLARDGLRYDSASDNVYMNAEQEEKAKKANNPQHSITKDEIKQYVKEYVQAAKNSIAAGADGVEIHSANGYLLNQFLDPHSNNRTDEYGGSIENRARFTLEVVDAVVDAIGPEKVGLRLSPYGVFNSMSGGAETGIVAQYAYVLGELERRAKAGKRLAFVHLVEPRVTNPFLTEGEGEYNGGSNKFAYSIWKGPIIRAGNFALHPEVVREEVKDPRTLIGYGRFFISNPDLVDRLEKGLPLNKYDRDTFYKMSAEGYIDYPTYEEALKLGWDKN

SEQ ID NO: 13 - DNA, 1017 bp, Bacillus subtilis
  CDS (1)..(1017)
atggccagaaaattatttacacctattacaattaaagatatgacgttaaaaaaccgcattgtcatgtcgccaatgtgcatgtattcttctcatgaaaaggacggaaaattaacaccgttccacatggcacattacatatcgcgcgcaatcggccaggtcggactgattattgtagaggcgtcagcggttaaccctcaaggacgaatcactgaccaagacttaggcatttggagcgacgagcatattgaaggctttgcaaaactgactgagcaggtcaaagaacaaggttcaaaaatcggcattcagcttgcccatgccggacgtaaagctgagcttgaaggagatatcttcgctccatcggcgattgcgtttgacgaacaatcagcaacacctgtagaaatgtcagcagaaaaagtaaaagaaacggtccaggagttcaagcaagcggctgcccgcgcaaaagaagccggctttgatgtgattgaaattcatgcggcgcacggatatttaattcatgaatttttgtctccgctttccaaccatcgaacagatgaatatggcggctcacctgaaaaccgctatcgtttcttgagagagatcattgatgaagtcaaacaagtatgggacggtcctttatttgtccgtgtatctgcttctgactacactgataaaggcttagacattgccgatcacatcggttttgcaaaatggatgaaggagcagggtgttgacttaattgactgcagctcaggcgcccttgttcacgcagacattaacgtattccctggctatcaggtcagcttcgctgagaaaatccgtgaacaggcggacatggctactggtgccgtcggcatgattacagacggttcaatggctgaagaaattctgcaaaacggacgtgccgacctcatctttatcggcagagagcttttgcgggatccattttttgcaagaactgctgcgaaacagctcaatacagagattccggcccctgttcaatacgaaagaggctggtaa

SEQ ID NO: 14 - protein, 338 aa, Bacillus subtilis
MARKLFTPITIKDMTLKNRIVMSPMCMYSSHEKDGKLTPFHMAHYISRAIGQVGLIIVEASAVNPQGRITDQDLGIWSDEHIEGFAKLTEQVKEQGSKIGIQLAHAGRKAELEGDIFAPSAIAFDEQSATPVEMSAEKVKETVQEFKQAAARAKEAGFDVIEIHAAHGYLIHEFLSPLSNHRTDEYGGSPENRYRFLREIIDEVKQVWDGPLFVRVSASDYTDKGLDIADHIGFAKWMKEQGVDLIDCSSGALVHADINVFPGYQVSFAEKIREQADMATGAVGMITDGSMAEEILQNGRADLIFIGRELLRDPFFARTAAKQLNTEIPAPVQYERGW

SEQ ID NO: 15 - DNA, 37 bp, Artificial
  primer
aggaggaattaaccatgtcgtacatgaactttgaccc

SEQ ID NO: 16 - DNA, 22 bp, Artificial
  primer
ttagtacttcttttcctctttc

SEQ ID NO: 17 - DNA, 43 bp, Artificial
  primer
aggaggaattaaccatgtcgtttatgaactttgaaccaaagcc

SEQ ID NO: 18 - DNA, 27 bp, Artificial
  primer
ctatttcttgtaacccttggcaacagc

SEQ ID NO: 19 - DNA, 45 bp, Artificial
  primer
aggaggaattaaccatggcaactattttcgatccgatcaaactgg

SEQ ID NO: 20 - DNA, 31 bp, Artificial
  primer
ttacagcgtcgggtagtcgatgtagccgacc

SEQ ID NO: 21 - DNA, 35 bp, Artificial
  primer
aggaggaattaaccatgccgactcttttcgacccc

SEQ ID NO: 22 - DNA, 21 bp, Artificial
  primer
ctacttttggtcagcggtggg

SEQ ID NO: 23 - DNA, 40 bp, Artificial
  primer
aggaggaattaaccatgtcatctgaaaaactgtattcccc

SEQ ID NO: 24 - DNA, 40 bp, Artificial; identical to SEQ ID NO: 23
  primer

SEQ ID NO: 25 - DNA, 40 bp, Artificial
  primer
aggaggaattaaccatgccatttgttaaggactttaagcc

SEQ ID NO: 26 - DNA, 30 bp, Artificial
  primer
ttaatttttgtcccaaccgagttttagagc

SEQ ID NO: 27 - DNA, 37 bp, Artificial
  primer
aggaggaattaaccatggccagaaaattatttacacc

SEQ ID NO: 28 - DNA, 24 bp, Artificial
  primer
ttaccagcctctttcgtattgaac

SEQ ID NO: 29 - DNA, 27 bp, Artificial
  primer
ctcatatggcgacaatccgacctgacg

SEQ ID NO: 30 - DNA, 28 bp, Artificial
  primer
ctgcatgcttgttgtctgacagtgcgtc

SEQ ID NO: 31 - DNA, 1566 bp, Rhodococcus erythropolis
  CDS (1)..(1566)
atggcgacaatccgacctgacgacaaagcaatagacgccgccgcaaggcattacggcatcactctcgacaaaacagcccggctcgagtggccggcactgatcgacggagcactgggctcctacgacgtcgtcgaccagttgtacgccgacgaggcgaccccgccgaccacgtcacgcgagcacgcggtgccaagtgcgagcgaaaatcctttgagcgcttggtatgtgaccaccagcatcccgccgacgtcggacggcgtcctgaccggccgacgcgtggcgatcaaggacaacgtgaccgtggccggagttccgatgatgaacggatctcggacggtagagggatttactccgtcacgcgacgcgactgtggtcactcgactactggcggccggtgcaaccgtcgcgggcaaagctgtgtgtgaggacctgtgtttctccggttcgagcttcacaccggcaagcggaccggtccgcaatccatgggaccggcagcgcgaagcaggtggatcatccggcggcagtgcagcactcgtcgcaaacggtgacgtcgattttgccatcggcggggatcaaggcggatcgatccggatcccggcggcattctgcggcgtcgtcgggcacaagccgacgttcgggctcgtcccgtataccggtgcatttcccatcgagcgaacaatcgaccatctcggcccgatcacacgcacggtccacgatgcagcactgatgctctcggtcatcgccggccgcgacggtaacgacccacgccaagccgacagtgtcgaagcaggtgactatctgtccaccctcgactccgatgtggacggcctgcgaatcggaatcgttcgagagggattcgggcacgcggtctcacagcccgaggtcgacgacgcagtccgcgcagcggcacacagtctgaccgaaatcggttgcacggtagaggaagtaaacatcccgtggcatctgcatgctttccacatctggaacgtgatcgccacggacggtggtgcctaccagatgttggacggcaacggatacggcatgaacgccgaaggtttgtacgatccggaactgatggcacactttgcttctcgacgcattcagcacgccgacgctctgtccgaaaccgtcaaactggtggccctgaccggccaccacggcatcaccaccctcggcggcgcgagctacggcaaagcccggaacctcgtaccgcttgcccgcgccgcctacgacactgccttgagacaattcgacgtcctggtgatgccaacgctgccctacgtcgcatccgaattgccggcgaaggacgtagatcgtgcaaccttcatcaccaaggctctcgggatgatcgccaacacggcaccattcgacgtgaccggacatccgtccctgtccgttccggccggcctggtgaacgggcttccggtcggaatgatgatcaccggcagacacttcgacgatgcgacagtccttcgtgtcggacgcgcattcgaaaagcttcgcggcgcgtttccgacgccggccgaacgcgcctccaactctgcaccacaactcagccccgcctag

SEQ ID NO: 32 - protein, 521 aa, Rhodococcus erythropolis
MATIRPDDKAIDAAARHYGITLDKTARLEWPALIDGALGSYDVVDQLYADEATPPTTSREHAVPSASENPLSAWYVTTSIPPTSDGVLTGRRVAIKDNVTVAGVPMMNGSRTVEGFTPSRDATVVTRLLAAGATVAGKAVCEDLCFSGSSFTPASGPVRNPWDRQREAGGSSGGSAALVANGDVDFAIGGDQGGSIRIPAAFCGVVGHKPTFGLVPYTGAFPIERTIDHLGPITRTVHDAALMLSVIAGRDGNDPRQADSVEAGDYLSTLDSDVDGLRIGIVREGFGHAVSQPEVDDAVRAAAHSLTEIGCTVEEVNIPWHLHAFHIWNVIATDGGAYQMLDGNGYGMNAEGLYDPELMAHFASRRIQHADALSETVKLVALTGHHGITTLGGASYGKARNLVPLARAAYDTALRQFDVLVMPTLPYVASELPAKDVDRATFITKALGMIANTAPFDVTGHPSLSVPAGLVNGLPVGMMITGRHFDDATVLRVGRAFEKLRGAFPTPAERASNSAPQLSPA

SEQ ID NO: 33 - DNA, 945 bp, Ochrobactrum anthropi
  CDS (1)..(945)
atgtgcaataattgccattacaccattcacggccggcatcatcatttcggctgggacaactcgttccagccggctgaaacggtcgcgcccggctcgaccctgaaattcgaatgtctggacagcggcgcaggccactatcatcgcggcagcacagtcgccgatgtgtcgacgatggatttttccaaggtcaatccggttaccggccccatcttcgtcgatggagccaaaccgggcgatgtcctgaaaatcaccatccaccagttcgagccatcaggcttcggctggacggcaaatattccgggcttcggtcttctcgccgacgacttcaaggaaccggcgctagcattgtggaactacaatcccacaacgctggagccagcactcttcggagagcgtgcgcgcgtgccgctgaagccgttcgccggaaccatcggcgtcgcaccggcggaaaagggcctgcattcggtcgtaccaccgcgtcgtgtcggcggcaatctcgacatccgcgatcttgcagccggaaccacgctttatctgccgatcgaagtcgaaggcgctttgttctccattggtgatacccatgcggcacagggcgacggcgaagtgtgcggcaccgccatcgaaagcgcgatgaatgtcgctctgacgctggatctcatcaaggatacgccactgaagatgccccggttcaccacgccggggccagtgacgcggcacctcgataccaagggttacgaagtcaccaccggtatcgggtccgatctgtgggaaggcgcgaaagccgccctctccaacatgatcgaccttctttgccagacgcagaacctcaacccggtggatgcctatatgctctgctcggcctgcggtgatctgcgtatcagcgaaatcgtcgatcagccgaactgggtcgtatcgttctacttcccgcgttccgttttcgaataa

SEQ ID NO: 34 - protein, 314 aa, Ochrobactrum anthropi
MCNNCHYTIHGRHHHFGWDNSFQPAETVAPGSTLKFECLDSGAGHYHRGSTVADVSTMDFSKVNPVTGPIFVDGAKPGDVLKITIHQFEPSGFGWTANIPGFGLLADDFKEPALALWNYNPTTLEPALFGERARVPLKPFAGTIGVAPAEKGLHSVVPPRRVGGNLDIRDLAAGTTLYLPIEVEGALFSIGDTHAAQGDGEVCGTAIESAMNVALTLDLIKDTPLKMPRFTTPGPVTRHLDTKGYEVTTGIGSDLWEGAKAALSNMIDLLCQTQNLNPVDAYMLCSACGDLRISEIVDQPNWVVSFYFPRSVFE

SEQ ID NO: 35 - DNA, 1212 bp, Artificial
  codon optimised OYE gene from Candida macedoniensis
atgtcctacatgaactttgacccgaaaccgctgggggataccaacatcttcaaaccgattaaaatcggtaataacgaactgaagcaccgtgttgtaatgccggcgctgacgcgtatgcgtgcaattgccccgggtaacattccgaacaccgaatgggctgaagaatactatcgccagcgttctcagtacccgggcactctgatcatcaccgaaggcaccttcccgtccgcgcagtctggtggttatccgaatgttccgggtatttggtctaaggaacagctggctgaatggaagaaaatcttcaatgcgattcacgaaaacaaaagcttcgtttgggttcaactgtgggtgctgggccgtcaggcttggccggaagttctgaagaaagaagggctgcgttatgactccgcgaccgatgacctgtatatgggtgaggaagaaaaagaacgcgcactgaaagcaaacaacccacaacacgggattaccaaggaagaaattaaacagtacatcaaagagtatgttgatgcggcgaaaaaggctatcgacgccggtgctgacggcgtgcagatccattccgccaacggttacctgctgaaccagttcctggacccgatctccaataaccgtaccgatgaatacggtggctctatcgaaaatcgcgctcgtttcaccctggaagtagttgacgccgttgttgacgccgtgggcgcggagcgtacctccatccgcttctctccgtacggaacgttcggtactatgtccggaggcgagaacccgggaatcgtggctcaatacgcttacgttattggtgaactggagaaacgcgcacgcgctggtaaacgcctggcgttcatcgatctggtggaaccgcgcgttactgatccgttcctgccggagtttgaaaaatggtttaaagaaggtaccaacgagttcatctacagcatttggaaaggtccggtactgcgtgttggcaattatgccctggacccggaccaggcgactctggatagcaaaaagccgaacacgctgattggctatggtcgcagcttcatcgccaatccagacctggtgtaccgcctggaaaaaggactgcctctgaacaaatatgaccgcaacactttctacactttcactaaggaaggatacactgactacccgtcctacgaggaatctgtagcaaaagggtataaaaaggaagaaaagaaatactaa

SEQ ID NO: 36 - DNA, 1197 bp, Artificial
  codon optimised KYE1 gene from Kluyveromyces lactis NRRL Y-1140
atgagctttatgaatttcgagcctaaaccgctggccgatactgatatcttcaagccgatcaaaattggtaataccgagctgaaacaccgtgtagttatgccggctctgactcgtatgcgcgcgctgcatccaggtaacgtgccgaacccggactgggctgttgaatactaccgtcagcgttcccaatatcctggcaccatgatcatcactgaaggtgcttttccgtctgcccagtctggtggctacgataatgctcctggcgtttggtctgaggaacaactggcccagtggcgcaagatctttaaagcgatccatgacaacaaaagcttcgtctgggtacagctgtgggttctgggtcgtcaggcgttcgcagataacctggctcgcgacggtctgcgttacgattctgcctccgacgaagtttacatgggtgaagatgagaaagaacgcgccatccgttccaacaacccgcaacacggtatcacgaaagatgaaatcaagcagtacatccgtgactacgtggacgcagctaaaaagtgtatcgacgcgggcgctgatggtgttgaaattcactctgcaaacggctacctgctgaaccagtttctggaccctatctccaacaaacgtactgacgaatatggcggttctatcgagaatcgtgcccgcttcgtactggaagtagttgacgcggttgtggacgcggtgggcgcggagcgtaccagcattcgtttcagcccatatggcgtattcggtactatgtccggtgtttccgacccggttctggttgcacagttcgcatatgtcctggcggaactggaaaaacgtgcaaaagctggtaaacgtctggcatacgttgacctggtggaaccgcgtgtaacctccccattccagccggaatttgaaggctggtataaaggcggcaccaacgaattcgtatacagcgtttggaaaggtaacgttctgcgcgttggcaactacgctctggatccggacgctgcaattaccgactctaaaaacccgaataccctgatcggctacggccgtgctttcatcgctaaccctgatctggtagaacgtctggagaaaggcctgcctctgaaccagtacgatcgtccttccttttacaaaatgtccgccgagggttatatcgattatccgacttatgaagaagcggttgctaagggctataaaaagtaa

SEQ ID NO: 37 - DNA, 1050 bp, Artificial
  codon optimised xenB gene from Pseudomonas fluorescens I-C
atggcgactatttttgacccgattaaactgggagatctggaactgtctaaccgcattatcatggcgccgctgacccgttgccgtgcggatgagggccgcgttccaaatgctctgatggctgagtactatgtgcagcgtgcgtccgcaggtctgattctgtctgaagcgacctccgtgactcctatgggggtaggctacccggatacgccgggtatttggtccaatgaccaggtgcgtggatggacgaacatcaccaaagctgttcacgcagcaggcggtaagatcgttctgcaactgtggcacgttggccgtatttcccacccgctgtacctgaacggagaggcgccggtagctccgtctgctatccaaccgaaaggtcatgtatccctggtgcgtccgctggctgattatcctactccgcgtgcgctggaaaccgcggagattgcggaaatcgttgaagcatatcgtacgggagccgaaaacgcgaaagctgcgggctttgacggcgtcgaaatccatggcgctaacggttatctgctggatcagtttctgcaaagctccacgaatcagcgtaccgacaactacggcggatctctggaaaaccgtgcgcgcctgctgctggaagtaaccgacgccgcaattgatgtgtggggagccggccgtgttggtgttcacctggctcctcgtgcggatagccacgacatgggtgacgacaacctggcagaaaccttcacttatgtggctcgcgaactgggaaaacgcgggatcgcattcatctgttctcgcgaaaaagaaggtgccgactccctgggcccgcaactgaaggaagcctttggaggcgcttatattgcgaacgagcgttttaccaaagattctgcaaacgcgtggctggcggaaggtaaagcagatgcagttgcgtttggcgttccgttcattgccaaccctgacctgccggctcgcctgaaggcagacgctcctctgaacgaacctcgcccggaactgttctacggcaaaggtccggtgggatatatcgattacccgaccctgtaa

SEQ ID NO: 38 - DNA, 1083 bp, Artificial
  codon optimised ncr gene from Pseudomonas syringae pv. glycinea
atgccaactctgtttgacccgctgactctgggcgacctgcaatccccgaaccgtgttctgatggctcctctgacccgtggacgtgccacccgtgagcacgtgccgaccgagctgatgatcgaatactatacccagcgcgcgagcgcaggcctgatcatcaccgaagccacgggcatcacccaggaaggtctgggctggccgtatgctccggggatctggtccgacgagcaggtggaagcctggaaaccggtgactcaggctgtacatgaagcgggtggacgcattatcctgcaactgtggcacatgggtcgcaccgttcattcttctttcctgggtggcgcaaaacctgtttctagctccgcaacccgtgcaccgggacaggctcacacttacgaagggaaacaggattacgacgaagcgcgtccgctgagcgccgatgaaattccgcgtctgctgaacgattatgagcacgctgccaaaaacgcaatggcagcaggcttcgatggtgtccagattcacgccgctaacggctatctgatcgaccaatttctgcgtgataactccaacgttcgcggggacgcatacggtggctctatcgagaaccgtattcgcctgctggttgaagttacccgtcgtgttgccgaaaccgtgggagccgagaaaaccggtgtgcgtctgtccccgaatggcgactcccagggggtgaacgattctaacccagaaccgctgttttctgcggcggctaaagcactggatgagattggtatcgcccacctggaactgcgtgaaccgggctatgaaggcacttttggcaaagctgaccgtccgccggtacatccagtcatccgtcaggcattttctcgtaccctgatcctgaacagcgattacacgctggaaaccgcacaggctgcactggcaaccggtgaagctgatgctattacttttggccgtccgtttctggcgaaccctgacctgccgcatcgtttcgcggaacgtctgccgctgaacaaggatgttatggaaacctggtactctcagggaccggagggttacgtagattatccaaccgcagatcagaaataa

(19) United States
(12) Patent Application Publication
(10) Pub. No.: US 2011/0183386 A1
(43) Pub. Date: Jul. 28, 2011

(54) PREPARATION OF EPSILON-CAPROLACTAM FROM (Z)-6,7-DIHYDRO-1H-AZEPIN-2(5H)-ONE

(75) Inventors: Petronella Catharina Raemakers-Franken, Budel (NL); Martin Schurmann, Julich (DE); Axel Christoph Trefzer, Leidschendam (NL); Stefaan Marie Andre De Wildeman, Maasmechelen (BE)

(73) Assignee: DSM IP ASSETS B.V., Heerlen (NL)

(21) Appl. No.: 12/991,605

(22) PCT Filed: May 20, 2009
(86) PCT No.: PCT/NL2009/050273

§ 371 Date: Mar. 7, 2011

(30) Foreign Application Priority Data
May 20, 2008 (EP) 08156599.6

(51) Int. Cl. C12P 17/10; C12N 1/15; C12N 1/19; C12N 1/21; C07H 21/00

(52) U.S. Cl. 435/121; 435/254.3; 435/254.5; 435/254.21; 435/254.23; 435/254.22; 435/254.2; 435/252.31; 435/252.32; 435/252.33; 536/23.1

(57) ABSTRACT

The invention relates to a method for preparing ε-caprolactam comprising reducing the carbon-carbon double bond of (Z)-6,7-dihydro-1H-azepin-2(5H)-one, wherein the reduction is catalysed by a biocatalyst. The invention further relates to a novel host cell comprising a biocatalyst capable of catalysing said reduction and to a novel polynucleotide encoding a biocatalyst capable of catalysing said reduction.

The invention relates to a method for preparing ε-caprolactam from (Z)-6,7-dihydro-1H-azepin-2(5H)-one. The invention further relates to a host cell which may be used in the preparation of caprolactam.

Caprolactam is a lactam which may be used for the production of polyamide, for instance nylon-6 or nylon-6,12 (a copolymer of caprolactam and laurolactam). Various manners of preparing caprolactam from bulk chemicals are known in the art and include the preparation of caprolactam from cyclohexanone, toluene, phenol, cyclohexanol, benzene or cyclohexane. These intermediate compounds are generally obtained from mineral oil. In view of a growing desire to prepare materials using more sustainable technology it would be desirable to provide a method wherein caprolactam is prepared from an intermediate compound that can be obtained from a biological source or at least from an intermediate compound that is converted into caprolactam using a biochemical method. Furthermore, it would be desirable to provide a method that requires less energy than conventional chemical processes making used of bulk chemicals from petrochemical origin.

It is known to prepare caprolactam from 6-aminocaproic acid (6-ACA), e.g. as described in U.S. Pat. No. 6,194,572. As disclosed in WO 2005/068643, 6-ACA may be prepared biochemically by converting 6-aminohex-2-enoic acid (6-AHEA) in the presence of an enzyme having α,β-enoate reductase activity. The 6-AHEA may be prepared from lysine, e.g. biochemically or by pure chemical synthesis. Although, the preparation of 6-ACA via the reduction of 6-AHEA is feasible by the methods disclosed in WO 2005/068643, the inventors have found that—under the reduction reaction conditions—6-AHEA may spontaneously and substantially irreversibly cyclise to form an undesired side-product, notably β-homoproline. This cyclisation may be a bottle neck in the production of 6-ACA, and lead to a considerable loss in yield.

It is an object of the invention to provide a novel method for preparing caprolactam that can serve as an alternative for known methods. It is in particular an object to provide a novel method for preparing an intermediate compound that can be used to prepare caprolactam from.

It is a further object to provide a novel method that would overcome one or more of the drawbacks mentioned above.

It is a further object to provide a novel fermentative method for preparing caprolactam.

One or more further objects which may be solved in accordance with the invention, will follow from the description, below.

It has now been found possible to prepare caprolactam biocatalytically from a specific starting compound.

Accordingly, the present invention relates to a method for preparing ε-caprolactam comprising reducing the carbon-carbon double bond of (Z)-6,7-dihydro-1H-azepin-2(5H)-one, wherein the reduction is catalysed by a biocatalyst.

The invention is based on the insight that it is possible to prepare caprolactam biocatalytically from lysine or from a product that can be obtained by the cyclisation of lysine.

The invention allows the preparation of caprolactam, that is essentially free of undesired cyclic side-product, in particular β-homoproline.

In an advantageous embodiment of the invention caprolactam is prepared fermentatively.

The term “or” as used herein means “and/or” unless specified otherwise.

The term “a” or “an” as used herein means “at least one” unless specified other wise.

When referring to a noun (e.g. a compound, an additive etc.) in singular, the plural is meant to be included. Thus, when referring to a specific noun, e.g. “compound”, this means “at least one” of that noun, e.g. “at least one compound”, unless specified otherwise.

When referred to a compound of which stereisomers exist, the compound may be any of such stereoisomers or a combination thereof. Thus, when referred to, e.g., an amino acid of which enantiomers exist, the amino acid may be the L-enantiomer, the D-enantiomer or a combination thereof. In case a natural stereoisomer exists, the compound is preferably a natural stereoisomer.

When referring herein to carboxylic acids or carboxylates, e.g. 6-ACA, another amino acid or a fatty acid, these terms are meant to include the protonated carboxylic acid, their corresponding carboxylate (their conjugated bases) as well as salts thereof. When referring herein to amino acids, e.g. 6-ACA, this term is meant to include amino acids in their zwitterionic form (in which the amino group is in the protonated and the carboxylate group is in the deprotonated form), the amino acid in which the amino group is protonated and the carboxylic group is in its neutral form, and the amino acid in which the amino group is in its neutral form and the carboxylate group is in the deprotonated form, as well as salts thereof. Likewise, when referring to an amine (e.g. lysine or another amino acid, or ACL), this is meant to include the protonated amine (typically cationic, e.g. R—NH₃⁺) and the unprotonated amine (typically uncharged, e.g. R—NH₂).

When an enzyme is mentioned with reference to an enzyme class (EC) between brackets, the enzyme class is a class wherein the enzyme is classified or may be classified, on the basis of the Enzyme Nomenclature provided by the Nomenclature Committee of the International Union of Biochemistry and Molecular Biology (NC-IUBMB), which nomenclature may be found at http://www.chem.qmul.ac.uk/iubmb/enzyme/. Other suitable enzymes that have not (yet) been classified in a specified class but may be classified as such, are meant to be included.

The term “homologue” is used herein in particular for polynucleotides or polypeptides having a sequence identity of at least 30%, preferably at least 40%, more preferably at least 60%, more preferably at least 65%, more preferably at least 70%, more preferably at least 75%, more preferably at least 80%, in particular at least 85%, more in particular at least 90%, at least 91%, at least 92%, at least 93%, at least 94%, at least 95%, at least 96%, at least 97%, at least 98% or at least 99%. The term homologue is also meant to include nucleic acid sequences (polynucleotide sequences) which differ from another nucleic acid sequence (polynucleotide sequence) due to the degeneracy of the genetic code and encode the same polypeptide sequence.

Sequence identity or similarity is herein defined as a relationship between two or more polypeptide sequences or two or more nucleic acid sequences (polynucleotide sequences), as determined by comparing the sequences. Usually, sequence identities or similarities are compared over the whole length of the sequences, but may however also be compared only for a part of the sequences aligning with each other. In the art, “identity” or “similarity” also means the degree of sequence relatedness between polypeptide sequences or nucleic acid sequences (polynucleotide sequences), as the case may be, as determined by the match between strings of such sequences. Preferred methods to determine identity or similarity are designed to give the largest match between the sequences tested. In context of this invention a preferred computer program method to determine identity and similarity between two sequences includes BLASTP and BLASTN (Altschul, S. F. et al., J. Mol. Biol. 1990, 215, 403-410, publicly available from NCBI and other sources (BLAST Manual, Altschul, S., et al., NCBI NLM NIH Bethesda, Md. 20894). Preferred parameters for polypeptide sequence comparison using BLASTP are gap open 10.0, gap extend 0.5, Blosum 62 matrix. Preferred parameters for nucleic acid sequence comparison using BLASTN are gap open 10.0, gap extend 0.5, DNA full matrix (DNA identity matrix).

In a method of the invention, a biocatalyst is used, i.e. at least one reaction step in the method is catalysed by a biological material or moiety derived from a biological source, for instance an organism or a biomolecule derived there from. The biocatalyst may in particular comprise one or more enzymes. The biocatalyst may be used in any form. In an embodiment, one or more enzymes are used isolated from the natural environment (isolated from the organism it has been produced in), for instance as a solution, an emulsion, a dispersion, (a suspension of) freeze-dried cells, as a lysate, or immobilised on a support. In an embodiment, one or more enzymes form part of a living organism (such as living whole cells). The enzymes may perform a catalytic function inside the cell. It is also possible that the enzyme may be secreted into a medium, wherein the cells are present.

Living cells may be growing cells, resting or dormant cells (e.g. spores) or cells in a stationary phase. It is also possible to use an enzyme forming part of a permeabilised cell (i.e. made permeable to a substrate for the enzyme or a precursor for a substrate for the enzyme or enzymes).

A biocatalyst used in a method of the invention may in principle be any organism, or be obtained or derived from any organism. The organism may be eukaryotic or prokaryotic. In particular the organism may be selected from animals (other than humans, at least in as far as the use of the organism per se is involved), plants, bacteria, archaea, yeasts and fungi. A suitable biocatalyst or part thereof may in principle also be of human origin. In particular an enzyme may be obtained or derived from human cell material for use in method of the invention.

In an embodiment a biocatalyst, e.g. and enzyme, may originate from an animal, in particular from a part thereof—e.g. liver, pancreas, brain, kidney or other organ. The animal may in particular be selected from invertebrate marine animals, more in particular sponges (Porifera), in particular from Demospongiae, Pachastrellidae or Jaspidae, e.g. Jaspis sp., Pachastrella sp., Poecillastra sollasi, Choristidae and mammals, more in particular mammals selected from the group of Leporidae, Muridae, Suidae and Bovidae.

Suitable bacteria may in particular be selected amongst the group of Pseudomonas, Bacillus, Escherichia, Ochrobactrum, Citrobacter, Klebsiella, Mycobacterium, Providencia, Achromobacter, Rhodococcus, Myxococcus, Enterobacter, Methylophilus, Streptomyces, Achromobacter, Nocardia, Thermus and Alcaligenes.

Suitable fungi may in particular be selected amongst the group of Aspergillus, Tremella and Periconia.

Suitable yeasts may in particular be selected amongst the group of Candida, Saccharomyces, Kluyveromyces, Cryptococcus, and Trichosporon

It will be clear to the person skilled in the art that use can be made of a naturally occurring biocatalyst (wild type) or a mutant of a naturally occurring biocatalyst with suitable activity in a method according to the invention. Properties of a naturally occurring biocatalyst may be improved by biological techniques known to the skilled person in the art, such as e.g. molecular evolution or rational design. Mutants of wild-type biocatalysts can for example be made by modifying the encoding DNA of an organism capable of acting as a biocatalyst or capable of producing a biocatalytic moiety (such as an enzyme) using mutagenesis techniques known to the person skilled in the art (random mutagenesis, site-directed mutagenesis, directed evolution, gene recombination, etc.). In particular the DNA may be modified such that it encodes an enzyme that differs by at least one amino acid from the wild-type enzyme, so that it encodes an enzyme that comprises one or more amino acid substitutions, deletions and/or insertions compared to the wild-type, or such that the mutants combine sequences of two or more parent enzymes or by effecting the expression of the thus modified DNA in a suitable (host) cell. The latter may be achieved by methods known to the skilled person in the art such as codon optimisation or codon pair optimisation, e.g. based on a method as described in WO 2008/000632. WO 2003/010183 discloses a particularly suitable process for the preparation of variant polynucleotides using a combination of mutagenesis of a starting population of polynucleotides and recombination of the mutated polynucleotides.

A mutant biocatalyst may have improved properties, for instance with respect to one or more of the following aspects: selectivity towards the substrate, activity, stability, solvent tolerance, pH profile, temperature profile, substrate profile, susceptibility to inhibition, cofactor utilisation and substrate-affinity. Mutants with improved properties can be identified by applying e.g. suitable high through-put screening or selection methods based on such methods known to the skilled person in the art.

When referred to a biocatalyst, in particular an enzyme, from a particular source, recombinant biocatalysts, in particular enzymes, originating from a first organism, but actually produced in a (genetically modified) second organism, are specifically meant to be included as biocatalysts, in particular enzymes, from that first organism.

6,7-DAO used in a method according to the invention can in principle be obtained in any way. For instance, 6,7-DAO may be synthesised chemically or biocatalytically, e.g. microbiologically.

For instance, 6,7-DAO may be prepared based on a method as described by Donat and Nelson in J. Org. Chem. (1957), 22, 1106, of which the contents are incorporated by reference, in particular with respect to reaction conditions.

Further, the chemical preparation of 6,7-DAO may be based on, e.g., Reimschuessel, H. K. et al. J. Org. Chem. (1969), 34, 969, of which publication the contents are incorporated herein by reference, in particular with respect to reaction conditions. Based on this methodology, the skilled person will be able to prepare 6,7-DAO from ACL by diazotising ACL with NaNO₂ in the presence of HCl or HBr (or the like) by which the formed diazonium ACL derivative is transformed in situ to α-chloro- or α-bromocaprolactam, respectively. The latter compounds (or a similar compound If a different acid is used) can be converted into 6,7-DAO in an elimination reaction, using 2,6-lutidine as described in said reference.

In an embodiment, 6,7-DAO is prepared by converting α-amino-ε-caprolactam (ACL) into 6,7-DAO. 6,7-DAO can be prepared by removal of the α-amino group of ACL. In an embodiment this is accomplished by ammonia elimination. In another embodiment the removal comprises subsequent transamination, keto-group reduction and dehydration.

In a specific embodiment, the conversion of ACL to 6,7-DAO is carried out in the presence of a biocatalyst catalysing this conversion.

6,7-DAO may in particular be prepared from ACL in a method comprising biocatalytically removing the α-amino group from ACL by biocatalytic elimination of ammonia from ACL by a biocatalyst having ammonia lyase activity, thereby forming 6,7-DAO or removal of the α-aminogroup from ACL by another biocatalyst able of catalysing such elimination or another biocatalyst able of catalysing such removal of the ammonia group.

Removal of the α-amino group of ACL to yield 6,7-DAO may in particular be catalysed by a biocatalyst comprising a lyase (EC 4). Preferably, a C—N lyase (EC 4.3) is used, more preferably an ammonia lyase (EC 4.3.1) is used.

A biocatalyst catalysing the conversion of ACL to 6,7-DAO may for instance originate from an organism, as mentioned above.

It is also possible to select a suitable biocatalyst for the conversion of ACL to 6,7-DAO using a selection method, as described, next.

For instance, one may select for a biocatalyst using a library comprising a collection of potential biocatalysts for removal of the α-amino group from ACL. In a selection method for finding a suitable biocatalyst, the candidate biocatalysts are contacted with a culture medium wherein as a sole nitrogen source ACL and/or at least one functional analogue of ACL is present. Only those micro-organisms will be able to grow, which can use the ACL-analogue as a nitrogen source.

Thereafter, one or more samples are selected that show growth in such culture medium (the so called ‘growing cultures’). Thereafter, one or more of these growing cultures are tested for having activity towards converting ACL to 6,7-DAO. Optionally, in particular in case only one or more ACL-analogues have been used as the sole nitrogen source, the growing cultures are first checked for showing activity towards converting the ACL-analogue or analogues, after which one or more cultures showing such activity are tested for their activity towards converting ACL to 6,7-DAO.

Thus, in a specific aspect of the invention, the invention relates to a method of finding a biocatalyst capable of catalysing the removal of the α-amino group from ACL, comprising

providing a library comprising a plurality of candidate biocatalysts in one or more cell cultures, which cultures comprise a culture medium containing α-amino-ε-caprolactam and/or one or more analogues thereof as sole nitrogen source;

selecting one or more candidate biocatalysts which grow in said culture medium; and

screening for a biocatalyst which grows in said culture having catalytic activity in removing the α-amino group from ACL.

The term “selecting” as used herein is defined as a method in which one or more biocatalysts are tested for growth using certain specific conditions, which growth is an indication for the presence of the desired biocatalytic activity.

The term “screening” as used herein is defined as a method in which one or more biocatalysts are tested for one (or more) desired biocatalytic conversion(s).

The library may in particular be a metagenomic library, comprising genomic fragments of micro-organisms, which fragments may have been identified or which may be unidentified, and which fragments have been cloned into a suitable micro-organism for expression such as Escherichia, Pseudomonas, Bacillus, Streptomyces, or Saccharomyces. The fragments may in principle originate from any organism and one or more organisms. The organism(s) may be culturable or un-culturable under the existing conditions, may have a specific habitat, requiring specific environmental factors (e.g. temperature, pH, light, oxygen, nutrients) or symbiotic partners. In particular the organisms may be endosymbionts of a multicellular organism such as a sponge, insect, mammal or plant.

In an embodiment, the library comprises a variety of environmental samples containing candidate biocatalysts, in particular a variety of water samples (e.g. waste water samples), compost samples and/or soil samples. Such samples comprise a variety of wild-type micro-organisms.

The term “functional analogue of ACL” is used herein to indicate that the analogue comprises a functional group that may be recognised by the biocatalyst. In particular a functional analogue may have the L- or D-configuration or a mixture thereof in any ratio, consists of a seven-membered α-amino lactam or α-amino (thio)lactone with an additional carbon substituent at the α-position and optionally at the lactam nitrogen.

Preferably, an ACL-analogue is chosen which i) elicits the desired ACL ammonia lyase activity or alike activity leading to removal of the α-amino group from ACL and ii) has a low tendency towards eliciting side-reactions. In particular, the sole nitrogen source may consist of one or more compounds represented by formula I or II:

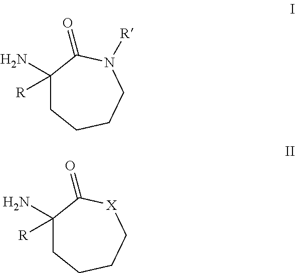

Herein, R and R′ independently represent a hydrogen atom, or an organic moiety—which optionally comprises of one or more heteroatoms. Heteroatoms in the organic moieties R and R′ may in particular be selected from N, S, O, F, Cl, Br, and I atoms. The organic moieties R and R′ may in particular be independently selected from substituted and unsubstituted C1-C6 alkyl groups. X represents an O atom or an S atom.

The use of one or more functional analogues as the sole nitrogen source is preferred because the inventors have contemplated that the chance of finding a false positive would be higher when using ACL.

Another suitable selection method for finding a biocatalyst capable of catalysing the conversion of ACL to 6,7-DAO is based on lysine auxotrophy complementation. Herein a suitable host cell, which is lysine auxotroph, and which contains ACL-hydrolase activity is used for expression screening of genomic or metagenomic libraries. Such a host cell may be naturally occurring or can be engineered e.g. by inactivating the lysA gene in E. coli and expressing a suitable ACL-hydrolase. Such a host cell is then used for constructing a library as described above resulting in various host cells containing different DNA fragments. Various cells comprise different cloned genes.

The host cells are contacted with a culture medium comprising 6,7-DAO as sole lysine precursor. Then, one or more host cells are selected which grow in this medium. Thereafter, one or more growing host cells are usually tested for having catalytic activity for the conversion of 6,7-DAO to ACL. Thereafter one or more growing host cells (usually selected from those having catalytic activity for the conversion of 6,7-DAO to ACL) are tested for having catalytic activity for the conversion of ACL to 6,7-DAO. A host cell having such activity can be used as a biocatalyst, or be used to obtain a biocatalyst therefrom.

Accordingly, the invention further relates to a method of detecting a biocatalyst capable of catalysing the removal of the α-amino group of α-amino-ε-caprolactam, comprising

providing lysine auxotrophic host cells, the host cells comprising a gene encoding an enzyme capable of catalysing the conversion of α-amino-ε-caprolactam into L-lysine, the host cells comprising a candidate gene encoding for an enzyme having lysine cyclase activity;

contacting the host cells with a library comprising various vectors containing a candidate gene encoding for an enzyme capable of catalysing the conversion of 6,7-DAO to ACL, whereby at least a part of the host cells are provided with said vector;

contacting the host cells, provided with said vector, with (Z)-6,7-dihydro-1H-azepin-2(5H)-one and an ammonia source;

selecting one or more cultures which grow in said culture medium; and

screening for one or more of said cultures which grow for having catalytic activity with respect to biocatalytic removal of the α-amino group of α-amino-ε-caprolactam, as the culture providing the biocatalyst capable of catalysing the elimination of the α-amino group of α-amino-ε-caprolactam.

Another suitable screening method contemplated by the inventors is based on using a molecular receptor and reporter system in a suitable host organism. Several such systems have been described in the art (Beggah, S.; Vogne, C.; Zenaro, E.; van der Meer, J. R. Microbial Biotechnology 2008, 1(1), 68-78; Sint Fiet, S.; van Beilen, J. B.; Witholt, B. Proceedings of the National Academy of Sciences 2006, 103(6), 1693-1698.). In such a system a suitable transcriptional regulator, herein also referred to as receptor, is able to bind a compound of interest such as 6,7-DAO or an analogue. Such a receptor may be a naturally occurring receptor having the desired properties in regards to e.g. specificity and binding affinity towards the compound of interest. In most cases these properties have to be optimized for the specific compound of interest and receptor interaction by protein engineering methods generally known in the art. Upon binding the receptor elicits transcription from a suitable promoter, which is linked to a suitable reporter gene, herein also referred to as reporter. Suitable reporters may in principle be a gene which elicits a detectibel, and preferably quantifiable, phenotype on the host strain such as production of a pigment, a fluorescent protein, an enzyme complementing an auxotrophy, or an antibiotic resistance marker.

Such a receptor/reporter system may be established in a host and subsequently be used for screening (e.g. if using a fluorescent protein such as a green fluorescent protein as reporter) or selection (e.g. if using an antibiotic resistance gene as reporter) of a suitable biocatalyst for conversion of ACL to 6,7-DAO. The host cells are contacted with a culture medium comprising ACL or an analogue thereof. Then, one or more host cell cultures are selected or screened for which elicit a phenotype corresponding to the expression of the reporter. Thereafter, one or more such host cell cultures are usually tested for having catalytic activity for the conversion of ACL to 6,7-DAO. A host cell having such activity can be used as a biocatalyst, or be used to obtain a biocatalyst therefrom.

Accordingly, the invention also relates to a method of finding a biocatalyst capable of catalysing the removal of the α-amino group of α-amino ε-caprolactam, comprising

identifying or engineering a receptor to specifically bind 6,7-DAO;

linking said receptor to a suitable reporter such as a β-galactosidase, green fluorescent protein, or an antibiotic resistance gene;

optionally optimising the binding of 6,7-DAO to the receptor via one or more rounds of protein engineering to obtain desired specificity (i.e. no or low signal from the natural ligand and/or ACL or analogues) and desired affinity towards 6,7-DAO or analogues thereof;

expressing such a receptor/reporter in a host suitable for metagenomic screening;

contacting the host cells with a library comprising various vectors containing a candidate gene encoding for a biocatalyst (such as an enzyme) capable of catalysing the conversion of 6,7-DAO to ACL, whereby at least a part of the host cells comprise said vector;

contacting the host cells, comprising said vector, with ACL or an analogue thereof;

selecting or screening for one or more cultures which show the desired phenotype based on expression of the chosen reporter; and

screening for one or more of said cultures for having catalytic activity with respect to biocatalytic removal of the α-amino group of α-amino-ε-caprolactam, as the culture providing the biocatalyst capable of catalysing the elimination of the α-amino group of α-amino-ε-caprolactam.

The gene encoding a biocatalyst, such as an enzyme, capable of catalysing the conversion of α-amino-ε-caprolactam into lysine may suitably be incorporated in the host cells using a vector, by conventional means.

The candidate gene encoding a biocatalyst having lysine cyclase activity may suitably be incorporated in the host cells using a vector, which may be the same or different as the vector encoding a biocatalyst capable of catalysing the conversion of α-amino-ε-caprolactam into lysine.

ACL, which may be used in a method according to the invention, can in principle be obtained in any way. For instance, ACL may be synthesised chemically, or biocatalytically. In an embodiment ACL is extracted from a natural source. For instance ACL may be obtained by, e.g. mild acidic, hydrolysis of an ACL moiety from a naturally occurring molecule comprising such moiety. For instance, naturally occurring molecules from which ACL may be obtained after hydrolysis are capuramycin from Streptomyces griseus; bengamide and isobengamide derivatives from sponges belonging to the Jaspidae; sesquiterpene derivatives from the sponge Poechillastra solassi; bengamide derivatives from Myxococcus virescens; caprolactin A and B from the deep sea isolate PC12/1000-B4 (Tetrahedron 1993, 49(30), 6569-6574); nocardiamycin derivatives from Nocardia sp.; and circinatin from the fungus Periconia circinata.

In an embodiment of the invention, ACL is prepared by cyclising lysine, which cyclisation is catalysed by a biocatalyst. In principle, D-lysine, L-lysine or a mixture thereof can be used. By cyclising these, D-ACL, L-ACL or a mixture thereof is formed. In practice, L-lysine is preferred.

A biocatalyst used in this cyclisation reaction, preferably comprises an enzyme having lysine cyclase activity. For instance an enzyme having lysine cyclase activity may be used originating from an organism as identified above.

In particular, an enzyme capable of catalysing the cyclisation of lysine to ACL, may be selected from the group of hydrolases (EC 3). The hydrolase preferably is selected from the group of hydrolases acting on ester bonds (esterases) (EC 3.1), and hydrolytic enzymes acting upon carbon-nitrogen bonds, other than peptide bonds (EC 3.5). An esterase may in particular be selected from the group of carboxylic ester hydrolases (EC 3.1.1) and more in particular carboxyl esterases (EC 3.1.1.1), preferably from pig liver esterases. An enzyme of EC class 3.5 may in particular be selected from the group of hydrolases mainly acting on linear amides (EC 3.5.1).

A hydrolase mainly acting on linear amides, such as an amidase, may in particular be such hydrolase from Ochrobactrum, Rhodococcus, Enterobacter, Thermus, Klebsiella, Aspergillus, Methylophilus or Mycobacterium. More in particular a hydrolase mainly acting on linear amides, such as an amidase, may be used from an organism of the group of Ochrobactrum anthropi, Rhodococcus erythropolis, Enterobacter cloacae, Thermus sp., Klebsiella terrigena, Klebsiella oxytoca, Aspergillus nidulans, Methylophilus methylotrophus and Mycobacterium smegmatis.

An amidase originating from Ochrobactrum anthropi NCIMB 40321 or an amidase originating from Rhodococcus erythropolis NCIMB 11540 is particularly advantageous in a method wherein ACL is further used for the preparation of caprolactam. Further, an amidase may be used as described in US 2005/0079595 or in EP-A 1 409 667 for the cyclisation reaction, the contents of which with respect to enzymes having lysine cyclase activity and genes coding for such enzymes are incorporated herein by reference.

Furthermore, an enzyme of EC class 3.5 may also in particular be selected from the group of hydrolases mainly acting on C—N bonds in cyclic amides (EC 3.5.2), which may also be referred to as a lactamase, and more in particular be selected from the group of lysine lactamases (EC 3.5.2.11). In particular, a lactamase (i.e. a hydrolase acting in cyclic amides) may be selected amongst L-lysine-1,6-lactam hydrolases (EC 3.5.2.11) and 6-aminohexanoate-cyclic dimer hydrolases (EC 3.5.2.12).

In an embodiment a lactamase, in particular an L-lysine lactamases, is selected amongst the group of lactamases from Aspergillus, Cryptococcus, Candida, Citrobacter, Trichosporon, Tremella and Providencia. More in particular, said lactamase may be selected amongst the group of lactamases originating from Aspergillus ustus, Aspergillus niger, Cryptococcus laurentii, Candida humicola, Citrobacter freundii, Trichosporon cutaneum, Tremella fuciformis, Tremella aurentia, Tremella foliacea, Tremella subanomalia and Providencia alcalifaciens.

In an embodiment a lactamase, in particular a 6-aminohexanoate-cyclic dimer hydrolase (EC 3.5.2.12) is a lactamase from Alcaligenes, such as from Alcaligenes lactamlytics or from Achromobacter, such as from Achromobacter xerosis.

A lipase may in particular be selected from lipases originating from a mammal, such as porcine lipase, bovine lipase or the like. In particular, a lipase used in a method of the invention may be a pancreatic lipase. Lipases are commercially available, e.g. porcine pancreas lipase may be obtained from Röhm (catalogue number 7023C) or from Sigma (catalogue number L-3126). It is known to the person skilled in the art that commercial pig liver esterase (PLE) preparations, e.g. available from Sigma, e.g. available from Sigma as a suspension (catalog number E2884) or in powder form (catalog number E3019), usually are a mixture of enzymes, amongst others, isoenzymes, of pig liver esterase. It is contemplated that one or more of these isoenzymes in the PLE preparation are responsible for the bioconversion of lysine to ACL. A person skilled in the art knows how to isolate, clone and/or express the pig liver esterase isoenzymes into a suitable host, if desired.

In an embodiment, one may use a non-ribosomal peptide synthase

(NRPS) for cyclisation of lysine. It is known for secondary metabolite producers to synthesise peptides via non-ribosomal peptide synthases (NRPSs). NRPSs are in detail described in, e.g., “Assembly-Line Enzymology for Polyketide and Nonribosomal Peptide Antibiotics”: Logic, Machinery, and Mechanisms Michael A. Fischbach and Christopher T. Walsh”, Chem. Rev. 2006, 106, 3468-3496, and in WO/00/58478. In some instances biocatalysts analogous to some parts of NRPSs are also used for production of modified amino acids (e.g. amino coumarin in e.g. novobiocin and β-hydroxy histidine as precursor for the imidazolone moiety in nikkomycin X) as building blocks for secondary metabolites. In bacteria and lower fungi biosynthetics genes required for production of secondary metabolites are typically clustered in one locus on the genome. In particular, in an embodiment wherein an NRPS is used, the NRPS may be a modular non-ribosomal peptide synthase comprising a lysine specific adenylation domain, a peptidyl carrier domain and a thioesterase/cyclisation domain.

In a specific embodiment a biocatalyst for cyclisation of lysine to ACL can be found in a gene cluster encoding the biosynthesis of bengamides, nocardiamycins, capuramycins, circinatins or any other ACL or ACL-derivative containing secondary metabolite. Such a gene cluster may be present in any microorganism producing such a compound or a microbial endosymbiont thereof. Such a gene cluster can readily be identified by methods generally known in the art such as genome scanning, whole genome sequencing, PCR using degenerated primers, or Southern hybridisation using information from known biosynthetic pathways. A specific biocatalyst may consist of a truncated NRPS module consisting of an adenylation domain specific for the activation of lysine, a peptidyl carrier domain, and a specific cyclisation domain. This cyclisation domain is expected to be homologous to known thioesterases catalysing the macrocyclisation of cyclic non-ribosomal peptides such as e.g. tyrocidin. It is expected that a cyclisation domain specific for cyclisation of lysine contains specific signature motifs allowing its differentiation from other cyclising thioesterases or thioesterase domains. The domain required for cyclisation of lysine may be encoded by one open reading frame resulting in a modular biocatalyst or in separate open reading frames resulting in separate proteins, which together form the biocatalyst. In the present invention use of such a biocatalyst may be advantageous, since the reaction is coupled to the hydrolysis of ATP and thus (at least substantially) irreversible.

In an embodiment, ACL is prepared by chemically converting lysine. This may for instance be accomplished by esterifying lysine with an alcohol, such as methanol, in the presence of thionyl chloride and neutralising the resultant reaction mixture with a base, such as sodium methoxide, whereby cyclisation occurs, e.g. as described in the Examples. It has been reported in Tetrahedron Lett. 1980, 21, 2443-2446 that L-lysine may be cyclised to form ACL, e.g. in refluxing toluene in the presence of a large excess of Al₂O₃. Alternatively, ACL may be formed by refluxing lysine and a hydroxide, such as NaOH, in a suitable alcohol (for instance 1-propanol, 1-butanol, 1-pentanol or 1-hexanol), e.g. in equimolar amounts, optionally in the presence of an excess of Al₂O₃.

As mentioned above, in a method according to the invention, ε-caprolactam is prepared by reducing the unsaturated carbon-carbon double bond of (Z)-6,7-dihydro-1H-azepin-2(5H)-one, yielding caprolactam.

Such reduction is carried out in the presence of a biocatalyst, capable of catalysing the reduction. Preferably such biocatalyst has reductase activity, in particular 6,7-DAO enone reductase activity, i.e. the catalyst is able to catalyse the reduction of the carbon-carbon double bond in 6,7-DAO, thereby forming caprolactam.

In particular, the biocatalyst may comprise an enzyme selected from the group of oxidoreductases (EC1), more in particular the oxidoreductase may be an oxidoreductase acting on the CH—CH group of donors (EC1.3) or an oxidoreductase that acts on NADH or NADPH (EC 1.6).

More specifically an oxidoreductase from EC 1.3.1 may be used, such as a 2-enone reductase (EC 1.3.1.33).

A specific example of class an EC 1.6 enzyme is old yellow enzyme 1 (OYE1) is EC 1.6.99.1. The biocatalyst for reducing 6,7-DAO may be used in combination with a cofactor, suitable cofactors are known in the art, depending on the biocatalyst (enzyme) that is used.

A biocatalyst capable of catalysing said reduction may originate from an organism such as mentioned above. In particular, said biocatalyst may originate from yeasts, plants, bacteria, archaea, fungi or mammals. More in particular a suitable biocatalyst capable of catalysing said reduction may originate from a micro-organism selected from Candida macedoniensis, Kluyveromyces lactis, Pseudomonas fluorescens, Pseudomonas syringae pv. glycinea, Escherichia coli, Saccharomyces cerevisiae and Bacillus subtilis.

In a specific embodiment, the biocatalyst for catalysing said reduction comprises an amino acid sequence as shown in any of the Sequence IDs 2, 4, 6, 8, 10, 12 or 14, or a homologue thereof.

Reaction conditions for any biocatalytic step in the context of the present invention may be chosen depending upon known conditions for the biocatalyst, in particular the enzyme, the information disclosed herein and optionally some routine experimentation.

In principle, the pH of the reaction medium used may be chosen within wide limits, as long as the biocatalyst is active under the pH conditions. Alkaline, neutral or acidic conditions may be used, depending on the biocatalyst and other factors. In case the method includes the use of a micro-organism, e.g. for expressing an enzyme catalysing a method of the invention, the pH is selected such that the micro-organism is capable of performing its intended function or functions. The pH may in particular be chosen within the range of four pH units below neutral pH and two pH units above neutral pH, i.e. between pH 3 and pH 9 in case of an essentially aqueous system at 25° C. A system is considered aqueous if water is the only solvent or the predominant solvent (>50 wt. %, in particular >90 wt. %, based on total liquids), wherein e.g. a minor amount of alcohol or another solvent (<50 wt. %, in particular <10 wt. %, based on total liquids) may be dissolved (e.g. as a carbon source) in such a concentration that micro-organisms which may be present remain active. In particular in case a yeast and/or a fungus is used, acidic conditions may be preferred, in particular the pH may be in the range of pH 3 to pH 8, based on an essentially aqueous system at 25° C. If desired, the pH may be adjusted using an acid and/or a base or buffered with a suitable combination of an acid and a base.

In principle, the incubation conditions can be chosen within wide limits as long as the biocatalyst shows sufficient activity and/or growth. Conditions may be selected from the group of aerobic, oxygen limited and anaerobic conditions.

Anaerobic conditions are herein defined as conditions without any oxygen or in which substantially no oxygen is consumed by the biocatalyst, in particular a micro-organism, and usually corresponds to an oxygen consumption of less than 5 mmol/l·h, in particular to an oxygen consumption of less than 2.5 mmol/l·h, or less than 1 mmol/l·h.

Aerobic conditions are conditions in which a sufficient level of oxygen for unrestricted growth is dissolved in the medium, able to support a rate of oxygen consumption of at least 10 mmol/l·h, more preferably more than 20 mmol/l·h, even more preferably more than 50 mmol/l·h, and most preferably more than 100 mmol/l·h.

Oxygen-limited conditions are defined as conditions in which the oxygen consumption is limited by the oxygen transfer from the gas to the liquid. The lower limit for oxygen-limited conditions is determined by the upper limit for anaerobic conditions, i.e. usually at least 1 mmol/l·h, and in particular at least 2.5 mmol/l·h, or most specifically at least 5 mmol/l·h. The upper limit for oxygen-limited conditions is determined by the lower limit for aerobic conditions, i.e. less than 100 mmol/l·h, less than 50 mmol/l·h, less than 20 mmol/l·h, or less than to 10 mmol/l·h.

Whether conditions are aerobic, anaerobic or oxygen limited is dependent on the conditions under which the method is carried out, in particular by the amount and composition of ingoing gas flow, the actual mixing/mass transfer properties of the equipment used, the type of micro-organism used and the micro-organism density.

In principle, the temperature used is not critical, as long as the biocatalyst, in particular the enzyme, shows substantial activity. Generally, the temperature may be at least 0° C., in particular at least 15° C., more in particular at least 20° C. A desired maximum temperature depends upon the biocatalyst. In general such maximum temperature is known in the art, e.g. indicated in a product data sheet in case of a commercially available biocatalyst, or can be determined routinely based on common general knowledge and the information disclosed herein. The temperature is usually 90° C. or less, preferably 70° C. or less, in particular 50° C. or less, more in particular or 40° C. or less.

In particular if a biocatalytic reaction is performed outside a host organism, a reaction medium comprising an organic solvent may be used in a high concentration (e.g. more than 50 wt. %, or more than 90 wt. %, based on total liquids), in case an enzyme is used that retains sufficient activity in such a medium.

In an advantageous method caprolactam is prepared making use of a whole cell biotransformation of the substrate for caprolactam or an intermediate for forming caprolactam (ACL, 6,7-DAO), comprising the use of a micro-organism wherein a lysine cyclase, and an ammonia lyase and/or biocatalyst with activity for removal the α-amino group from ACL, and a 6,7-DAO enone reductase and/or other biocatalyst capable of reducing 6,7-DAO to caprolactam are produced, and a carbon source for the micro-organism.

The carbon source may in particular contain at least one compound selected from the group of monohydric alcohols, polyhydric alcohols, carboxylic acids, carbon dioxide, fatty acids, glycerides, including mixtures comprising any of said compounds. Suitable monohydric alcohols include methanol and ethanol, Suitable polyols include glycerol and carbohydrates. Suitable fatty acids or glycerides may in particular be provided in the form of an edible oil, preferably of plant origin.

In particular a carbohydrate may be used, because usually carbohydrates can be obtained in large amounts from a biologically renewable source, such as an agricultural product, preferably an agricultural waste-material. Preferably a carbohydrate is used selected from the group of glucose, fructose, sucrose, lactose, saccharose, starch, cellulose and hemi-cellulose. Particularly preferred are glucose, oligosaccharides comprising glucose and polysaccharides comprising glucose.

The 6,7-DAO concentration may be within wide limits. A preferred concentration may inter alia depend on the biocatalyst that is used. Also, a preferred concentration for a method wherein both the preparation of 6,7-DAO and its conversion into caprolactam take place biocatalytically in the same cell (in an intracellular cascade reaction) or wherein both the preparation of 6,7 DAO and its conversion both take place in a one-pot type of process making use of enzymes (outside a cell) catalysing said reactions may be different from a method wherein 6,7-DAO has been prepared without using a biocatalyst or wherein 6,7-DAO has been in a different reactor.

It is contemplated that the 6,7-DAO concentration may be in the nanomolar range (1-1000 nmol/l), the micromolar range (1-1000 μmol/l) or the mmol/l range (1-1000 mmol), or in a concentration exceeding 1 mol/l.

In particular, in case preparation and conversion of 6,7-DAO take place intracellularly in the same cell or extracellularly in one a pot-type process, a concentration of 1 nmol/l or more, 100 nmol/l or more, 1 μmol/l or more, 10 μmol/l or more, or 100 μmol/l or more may already provide 6,7-DAO in a sufficient concentration for acceptable or advantageous conversion rates. In case the preparation of 6,7-DAO takes place intracellularly in the same cell as the conversion thereof, said concentrations in particular may be the intracellular concentration of 6,7-DAO. Extracellular concentrations of 6,7-DAO may be considerably lower in such embodiment; even 0 (i.e. below detection limit).

In case 6,7-DAO is converted inside an organism, but the preparation of 6,7-DAO has taken place outside that organism, or in case the preparation of 6,7-DAO has taken place in a different reaction system and for the 6,7-DAO conversion to caprolactam use is made of an enzyme isolated from an organism, the concentration of 6,7-DAO usually is at least 1 μmol/l, in particular at least 100 μmol/l, more in particular at least 1 mmol/l or at least 10 mmol/l (extracellular concentration in the medium wherein the organism is present if an organism is used; or concentration in the reaction medium wherein 6,7-DAO is converted in case an enzyme is used isolated from an organism).

The upper limit for the 6,7-DAO concentration is not particularly critical. The 6,7-DAO concentration may be exceeding 1 mol/l, 1 mol/l or less, in particular 0.5 mol/l or less or 0.1 mol/l or less. As will be understood by the skilled person in case a biocatalytical cell is used, especially a living cell, the 6,7-DAO concentration is usually chosen that the concentration is not toxic to the cell, at least not to the extent that its biocatalytic functioning is detrimentally affected to an unacceptable level.

A cell, in particular a recombinant cell, comprising one or more enzymes for catalysing a reaction step in a method of the invention can be constructed using molecular biological techniques, which are known in the art per se. For instance, if one or more biocatalysts are to be produced in a recombinant cell (which may be a heterologous system), such techniques can be used to provide a vector which comprises one or more genes encoding one or more of said biocatalysts. One or more vectors may be used which each comprise one or more genes. One or more vectors may be used, each vector comprising one or more of such genes. Such vector can comprise one or more regulatory elements, e.g. one or more promoters, which may be operably linked to a gene encoding a biocatalyst.

As used herein, the term “operably linked” refers to a linkage of polynucleotide elements (or coding sequences or nucleic acid sequence) in a functional relationship. A nucleic acid sequence is “operably linked” when it is placed into a functional relationship with another nucleic acid sequence. For instance, a promoter or enhancer is operably linked to a coding sequence if it affects the transcription of the coding sequence.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skilled in the art to act directly or indirectly to regulate the amount of transcription from the promoter. A “constitutive” promoter is a promoter that is active under most environmental and developmental conditions. An “inducible” promoter is a promoter that is active under environmental or developmental regulation. The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain.

The promoter that could be used to achieve the expression of the nucleic acid sequences coding for a biocatalyst for use in a method of the invention, in particular a 6,7-DAO enone reductase, and optionally at least one biocalalyst selected from the group of ammonia lyases and lysine cyclases, such as described herein above may be native to the nucleic acid sequence (nucleotide sequence) coding for the biocatalyst to be expressed, or may be heterologous to the nucleic acid sequence (coding sequence) to which it is operably linked. Preferably, the promoter is homologous, i.e. endogenous to the host cell.

If a heterologous promoter (to the nucleic acid sequence encoding the biocatalyst of interest) is used, the heterologous promoter is preferably capable of producing a higher steady state level of the transcript comprising the coding sequence (or is capable of producing more transcript molecules, i.e. mRNA molecules, per unit of time) than is the promoter that is native to the coding sequence. Suitable promoters in this context include both constitutive and inducible natural promoters as well as engineered promoters, which are known to the person skilled in the art.

A “strong constitutive promoter” is a promotor which causes mRNAs to be initiated at high frequency compared to a native host cell. Examples of such strong constitutive promoters in Gram-positive micro-organisms include SP01-26, SP01-15, veg, pyc (pyruvate carboxylase promoter), and amyE.

Examples of inducible promoters in Gram-positive micro-organisms include, the IPTG inducible Pspac promoter, the xylose inducible PxylA promoter.

Examples of constitutive and inducible promoters in Gram-negative microorganisms include, but are not limited to, tac, tet, trp-tet, Ipp, lac, lpp-lac, lacIq, T7, T5, T3, gal, trc, ara (P_{BAD}), SP6, λ-P_{R}, and λ-P_{L}.

Promoters for (filamentous) fungal cells are known in the art and can be, for example, the glucose-6-phosphate dehydrogenase gpdA promoters, protease promoters such as pepA, pepB, pepC, the glucoamylase glaA promoters, amylase amyA, amyB promoters, the catalase catR or catA promoters, glucose oxidase goxC promoter, beta-galactosidase lacA promoter, alpha-glucosidase aglA promoter, translation elongation factor tefA promoter, xylanase promoters such as xlnA, xlnB, xlnC, xlnD, cellulase promoters such as eglA, eglB, cbhA, promoters of transcriptional regulators such as areA, creA, xlnR, pacC, prtT, etc or any other, and can be found among others at the NCBI website (http://www.ncbi.nlm.nih.gov/entrez/).

The term “heterologous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous nucleic acids or proteins are not endogenous to the cell into which it is introduced, but has been obtained from another cell or synthetically or recombinantly produced. Generally, though not necessarily, such nucleic acids encode proteins that are not normally produced by the cell in which the DNA is transcribed or expressed. Similarly exogenous RNA encodes for proteins not normally expressed in the cell in which the exogenous RNA is present. Heterologous nucleic acids and proteins may also be referred to as foreign nucleic acids or proteins. Any nucleic acid or protein that one of skill in the art would recognize as heterologous or foreign to the cell in which it is expressed is herein encompassed by the term heterologous nucleic acid or protein.

A method according to the invention may be carried out in a host organism, which may be novel. Accordingly, the invention also relates to a novel host cell comprising one or more biocatalysts capable of catalysing the reduction of the carbon-carbon double bond of 6,7-DAO. The invention further relates a novel polynucleotide encoding a biocatalyst suitable for use in a method of the invention. In particular, the polynucleotide may comprise a nucleic acid sequence as defined in any of the Sequence IDs 35-38 or a non-wild type functional analogue thereof.

Functional analogues of a particular nucleotides sequence, as referred to herein, are in particular nucleotide sequences encoding the same amino acid sequence as that particular nucleotide sequence or encoding a homologue of that particular nucleotide sequence. In particular, preferred functional analogues are nucleotide sequence having a similar, the same or a better level of expression in a host cell of interest as the nucleotide sequence of which it is referred to as being a functional analogue of.

A polynucleotide comprising a nucleic acid sequence as shown in any of the Sequence IDs 35-38, has been found to show improved expression of the encoded biocatalyst compared to the wild-type gene in a suitable host cell, in particular E. coli.

A host cell according to the invention typically comprises one or more vectors comprising one or more genes encoding one or more biocatalysts (in particular enzymes) capable of catalysing the reduction of the carbon-carbon double bond of 6,7-DAO.

One or more suitable genes for a host cell or vector according to the invention may in particular be selected amongst genes encoding a biocatalyst (such as an enzyme) as mentioned herein above. In a specific embodiment, the cell or vector comprises a nucleic acid sequence encoding a biocatalyst comprising an amino acid sequence represented by Sequence ID 2, 4, 6, 8, 10, 12, 14 or a homologue thereof. Possible nucleic acid sequences encoding said sequences are shown in Sequence ID 1, 3, 5, 7, 9, 11 and 13, respectively. Preferred sequences include the nucleic acid sequences selected from the group of Sequence ID 35-38 and non-wild type functional analogues thereof.

A host cell according to the invention comprises at least one recombinant vector comprising a nucleic acid sequence encoding a biocatalyst (in particular an enzyme) with 6,7-DAO enone reductase activity. Optionally, the cell comprises a nucleic acid sequence encoding a biocatalyst (in particular an enzyme) with ACL ammonia lyase activity. In a specific embodiment a recombinant vector comprising a nucleic acid sequence encoding a biocatalyst (in particular an enzyme) with ACL ammonia lyase activity, which sequence can be in the same or a different vector as the sequence encoding the biocatalyst having 6,7-DAO enone reductase activity is present.

In an embodiment, a host cell according to the invention comprises at least one nucleic acid sequence encoding a biocatalyst (in particular an enzyme) with L-lysine cyclase activity. In a specific embodiment a recombinant vector comprising a nucleic acid sequence encoding a biocatalyst (in particular an enzyme) with L-lysine cyclase activity is present, which sequence can be in the same or a different vector as the sequence encoding the biocatalyst with 6,7-DAO enone reductase activity. Such gene may in particular comprise a nucleic acid sequence encoding a biocatalyst represented by Sequence ID 32, Sequence ID 34, or a homologue of any of these sequences. Examples of suitable nucleic acid sequences are given in Sequence ID 31 and Sequence ID 33.

A cell of the invention comprising a nucleic acid sequence encoding a biocatalyst with 6,7-DAO enone reductase activity, a nucleic acid sequence encoding a biocatalyst with ammonia lyase activity, and a nucleic acid sequence encoding a biocatalyst with lysine cyclase activity, is particularly suitable for a method wherein caprolactam is prepared from lysine, wherein purely chemical (i.e. not biocatalysed) reaction steps are avoided are at least considerably reduced. Thus, the cell may be used as a biocatalyst for all reaction steps to prepare caprolactam from lysine, which steps may take place intracellularly in at least some embodiments. Such as cell may be a natural micro-organism or a recombinant organism. In the recombinant organism at least one, at least two or at least three recombinant nucleic acid sequences are present for encoding any of said biocatalysts (usually enzymes).

The host cell may for instance be selected from the group of bacteria, yeasts and fungi. In particular the host cell may be selected from the genera selected from the group of Aspergillus, Penicillium, Saccharomyces, Kluyveromyces, Pichia, Candida, Hansenula, Bacillus, Corynebacterium, Pseudomonas, Gluconobacter and Escherichia, in which one or more encoding nucleic acid sequences as mentioned above have been cloned and expressed.

In particular, the host cell may be selected from the group of Escherichia coli, Bacillus subtilis, Corynebacterium glutamicum, Aspergillus niger, Penicillium chrysogenum, Saccharomyces cervisiae, Hansenula polymorpha, Candida albicans, Kluyveromyces lactis, Pichia stipitis and Pichia pastoris host cells. In a preferred embodiment, the host cell is capable of producing lysine (as a precursor).

The host cell may be in principle a naturally occurring organism or may be an engineered organism. Such an organism can be engineered using a mutation screening or metabolic engineering strategies known in the art. For instance such a host cell may be selected of the genus Corynebacterium, in particular C. glutamicum, enteric bacteria, in particular Escherichia coli, Bacillus, in particular B. subtilis and B. methanolicus, and Saccharomyces, in particular S. cerevisiae. Particularly preferred are C. glutamicum or B. methanolicus strains which have been developed for the industrial production of lysine.

In a specific embodiment, the host cell naturally comprises (or is capable of producing) one or more of the enzymes suitable for catalysing a reaction step in a method of the invention.

The invention will now be illustrated by the following examples.

### EXAMPLES

### General:

Molecular and Genetic Techniques

Standard genetic and molecular biology techniques are generally known in the art and have been previously described (Maniatis et al. 1982 “Molecular cloning: a laboratory manual”. Cold Spring Harbor Laboratory, Cold Spring Harbor, N.Y.; Miller 1972 “Experiments in molecular genetics”, Cold Spring Harbor Laboratory, Cold Spring Harbor; Sambrook and Russell 2001 “Molecular cloning: a laboratory manual” (3rd edition), Cold Spring Harbor Laboratory, Cold Spring Harbor Laboratory Press; F. Ausubel et al, eds., “Current protocols in molecular biology”, Green Publishing and Wiley Interscience, New York 1987).

Identification of Plasmids and Inserts

Plasmids carrying the different genes were identified by genetic, biochemical, and/or phenotypic means generally known in the art, such as resistance of transformants to antibiotics, PCR diagnostic analysis of transformant or purification of plasmid DNA, restriction analysis of the purified plasmid DNA or DNA sequence analysis.

### Example 1

### Biocatalytic Synthesis of ACL from Lysine

### 1.1 HPLC-MS Analysis for the Determination of Lysine and ACL

The calibration was performed by an external calibration line of both Lys and ACL. Lys elutes at a retention time (Rt) of 2.4 min (ESI(−)-MS, m/z 145) and ACL elutes at 4.4 min. (ESI(+)-MS, m/z 129).

The LC-UV-MS experiments were performed on an Agilent 1100, equipped with a quaternary pump, degasser, autosampler, column oven, diode-array detector (DAD) with 10-mm cell and a time-of-flight MS (Agilent, Waldbronn, Germany).

The LC-UV-MS conditions were:
Column: 50×4.6 mm Nucleosil C18, 5 μm (Machery & Nagel) precolumn coupled to a 250×4.6 mm id. Prevail C18, 5 μm (Alltech)
Eluent: 0.1(% v/v) formic acid in ultrapure water
Flow: 1 ml/min., before entering the MS the flow is split 1:3
Gradient: No gradient
Injection volume: 5 μl
UV detection: no UV used for detection
MS detection: ESI-MS, using the negative mode at Rt 0-4 minutes and the positive mode at 4-10 minutes. The electrospray ionization (ESI) used the following conditions; m/z 50-3600, 175 V fragmentor, 350° C. drying gas temperature, 10 L N₂/min drying gas, 50 psig nebuliser pressure and 2.5 kV capillary voltage.

### 1.2 Construction of Biocatalyst

Isolation of Chromosomal DNA from R. erythropolis NCIMB11540

Chromosomal DNA from Rhodococcus erythropolis NCIMB 11540 was isolated following the general protocol of the QIAGEN Genomic DNA Handbook (QIAGEN, Hilden, Germany) for the isolation of chromosomal DNA from gram positive bacteria. The raw preparation was purified by using a QIAGEN Genomic-tip 500/G column (QIAGEN, Hilden, Germany) and the manufacturer's procedure.

PCR Amplification of the R. erythropolis Lysine Cyclase Gene:

The sequences of the primers used for amplification of the R. erythropolis NCIMB 11540 lysine cyclase PCR-reaction contained restriction sites (underlined) for NdeI (forward-primer) and SphI (reverse primer) to allow the subsequent cloning into plasmid pMS470Δ8 (Balzer et al., Nucleic Acids Research, 1992, 20 (8): 1851-1858).

R. erythropolis -forward [SEQ ID No. 29]:
5′- CTCATATGGC GACAATCCGA CCTGACG -3′
R. erythropolis -reverse [SEQ ID No. 30]:
5′- CTGCATGCTT GTTGTCTGAC AGTGCGTC -3′

Synergy®-polymerase (GeneCraft, Cologne, Germany) was used according to the supplier's manual to allow TA-cloning of PCR-products. The PCR temperature profile was as follows: 1) 15 min 95° C.; 2) 1 min 94° C., 0.5 min 60° C., 4 min 72° C. (30×); 3) 10 min 72° C. The product of the PCR-reaction formed a clear band of the expected size on the analytical agarose gels.

Cloning of the PCR-Product into pCR®II-Vector (Invitrogen)

15 μl of the PCR product were purified by preparative agarose gel electrophoresis using the QIAquick gel extraction kit (QIAGEN, Hilden, Germany). 2 μl DNA-solution served as insert for the Invitrogen TA-Topo cloning procedure into the pCR®II plasmid with subsequent transformation of E. coli Top10F′. Positive clones were selected by white/blue screening on LB/ampicilline/IPTG/X-Gal plates. White colonies were picked and struck out for plasmid isolation. Restriction analysis with EcoRI showed clone pCR-33/3/1 to carry an insert of the desired size. DNA sequencing with M13f(−20) and M13rev-primers confirmed that the right fragment for the target lysine cyclase gene [SEQ ID. No. 31] coding for a lysine cyclase from Rhodococcus erythropolis NCIMB 11540 [SEQ ID No. 32] had been cloned.

Cloning of the pCR-33/3/1-Insert into pMS470Δ8

Plasmid pMS470Δ8 (Balzer et al., Nucleic Acids Research, 1992, 20 (8): 1851-1858) was isolated from E. coli by standard procedures. Double restriction with NdeI and SphI resulted in two fragments, from which the 4 kb part was eluted from an agarose gel. pCR-33/3/1 was digested with NdeI and SphI. A 1.6 kb fragment was isolated and purified using the QIAquick gel extraction kit (QIAGEN, Hilden, Germany). Ligation of the linearized pMS470 fragment and the SphI/NdeI gene fragment was performed with T4-DNA-ligase (Invitrogen) at 16° C. over night. Transformation of E. coli DH10B and restriction analysis of the plasmids of the ampicillin resistant clones with EcoRI resulted in a clone carrying the pMS470-33/3/1/11 plasmid.

Cultivation of E. coli DH10B pMS470-33/3/1/11-1

Fermentation for the production of the R. erythropolis NCIMB 11540 lysine cyclase was carried out on ten litre scale in an ISF-200 laboratory fermentor (Infors, Bottmingen, Switzerland). For the inoculation of the fermentor an over night (24 h) starter culture in 0.5 l Terrific Broth (TB; 12 g/l tryptone, 24 g/l yeast extract, 4 g/l glycerol, 2.31 g/l KH₂PO₄, 12.54 g/l K₂HPO₄, pH 7.0 containing 100 μg/ml carbenicillin) was used, which itself had been inoculated with 0.1 ml of the respective glycerol stock culture of E. coli DH10B pMS470-33/3/1/11-1.

The expression of R. erythropolis NCIMB 11540 lysine cyclase was induced by addition of 0.5 mM IPTG (final concentration) at a cell density of OD₆₂₀=0.8. After 20.5 hours of cultivation (OD₆₂₀=6.4) the cells were harvested by centrifugation (12 minutes at 12,227×g at 4° C.).

Preparation of Cell Free Extract of E. coli DH10B pMS470-33/3/1/11-1

The wet cells of E. coli DH10B pMS470-33/3/1/11-1 (117 g) were washed with 20 mM HEPES buffer (pH 7.0) and resuspended in 350 ml 0.1 M potassium phosphate buffer (pH 7.0). Cells were disrupted in a nanojet homogeniser (Haskel, Wesel, Germany) at 1300 bar and subsequently centrifuged (32,000×g for 60 min at 4° C.) to obtain the cell-free extracts (supernatant). The cell free extract was frozen in 10 ml portions and stored at −20° C. until further use.

Fermentation of E. coli Expressing the Nucleic Acid Sequence as Presented in [SEQ ID No. 33]

Escherichia coli cells expressing the nucleic acid sequence as presented in [SEQ ID. 33] encoding lysine cyclase as presented in [SEQ ID No. 34] were fermented as described in U.S. Pat. No. 7,241,602, whereby feed profile used to introduce feed 1 was used as described in Table 1 of U.S. Pat. No. 7,241,602.

Preparation of Enzyme Solution LAM0011 from Cells of E. coli

Enzyme solution LAM0011 containing lysine cyclase as presented in [SEQ ID No. 34] from cells of E. coli expressing the nucleic acid sequence as presented in [SEQ ID No. 33] was prepared as described in U.S. Pat. No. 7,241,602.

1.3 Biocatalytic Synthesis of ACL from Lysine

A substrate solution of 70 mM L-lysine.HCl and 1 mM ZnSO₄ in 100 mM sodium phosphate buffer (pH 7.0, containing 1 mM ZnSO₄) was prepared. To start the reaction, 1 ml of the cell free extract of E. coli DH10B pMS470-33/3/1/11-1 or 1 ml of enzyme solution LAM0011 were added to 9 ml substrate solution. Reaction mixtures were incubated on a shaker at 37° C. for 96 h. Furthermore, a chemical blank mixture (without cell free extract) and a biological blank (consisting of 1 ml cell free extract of E. coli DH10B pMS470-33/3/1/11-1 or 1 ml enzyme solution LAM0011 added to 9 ml 50 mM sodium phosphate buffer, pH 7.0 without L-lysine.HCl) were incubated under the same conditions. Samples were taken after 96 hours of incubation and analysed by HPLC-MS. The results are summarised in the following table.

TABLE 1
ACL formation from L-lysine in the presence
of enzyme solution LAM0011 and cell free extract
of E. coli DH10B pMS470-33/3/1/11-1

| Biocatalyst | ACL concentration [mg/kg] |
|---|---|
| Enzyme solution LAM0011 | 4.2 |
| Cell free extract of E. coli DH10B | 0.2 |
| pMS470-33/3/1/11-1 | |

It is shown that the formation ACL from L-lysine is catalysed by each of the biocatalysts mentioned in Table 1. No ACL was detected in the chemical and biological blank samples.

### Example 2

### Biocatalytic Synthesis of Caprolactam from 6,7-DAO

Plasmids and Strains

pBAD/Myc-His C was obtained from Invitrogen (Carlsbad, Calif., USA). Plasmid pBAD/Myc-His-DEST constructed as described in WO2005/068643, was used for protein expression. E. coli TOP10 (Invitrogen, Carlsbad, Calif., USA) was used for all cloning procedures and for expression of target genes.

Media

2*TY medium (16 g/l tryptone, 10 g/l yeast extract, 5 g/l NaCl) was used for growth of E. coli. Antibiotics (100 μg/ml carbenicillin, 25 μg/ml kanamycin) were supplemented to maintain plasmids. For induction of gene expression under control of the P_{BAD} promoter in pBAD/Myc-His-DEST derived plasmids, L-arabinose was added to final concentration of 0.02 to 0.2% (w/v).

### 2.1 HPLC-UV-MS Analysis for the Determination of 6,7-DAO and Caprolactam

### Calibration:

The calibration was performed by an external calibration line of both caprolactam and 6,7-DAO. Caprolactam elutes at retention time 24 min. (m/z 114) and 6,7-DAO elutes at 23 min. (m/z 112)

The LC-UV-MS experiments were perfomed on an Agilent 1100, equipped with a quaternary pump, degasser, autosampler, column oven, diode-array detector (DAD) with 10-mm cell and a single-quadrupole MS (Agilent, Waldbronn, Germany). The LC-UV-MS conditions are:
Column: 250×*4 mm Prevail column at 40° C. (Alltech, USA)
Eluent: A=0.1(% v/v) formic acid in ultrapure water
B=Acetonitrile (pa, Merck)
Flow: 1 ml/min., before entering the MS the flow is split 1:3
Gradient: The gradient was started at t=0 minutes with 100% (v/v) A, stayed there for 8 minutes and changed in 12 minutes to 95% (v/v) B (t=20 minutes).
From 20 to 21 minutes the gradient was held to 95% (v/v) B.
Injection volume: 5 μl
UV detection: λ=210, 220 and 250 nm
MS detection: ESI(+)-MS
The electrospray ionization (ESI) ran in the positive scan mode with the following conditions; m/z 50-1500, 50 V fragmentor, 0.1 m/z step size, 350° C. drying gas temperature, 10 L N₂/min drying gas, 50 psig nebuliser pressure and 2.5 kV capillary voltage.

### 2.2 Construction of Biocatalyst

Design of Expression Constructs

attB sites were added to all genes upstream of the ribosomal binding site and start codon and downstream of the stop codon to facilitate cloning using the Gateway technology (Invitrogen, Carlsbad, USA).

Cloning by PCR

The OYE gene (AB126227) from Candida macedoniensis AKU4588 [SEQ ID No. 1] encoding the amino acid sequence of the old yellow enzyme OYE of C. macedoniensis AKU4588 [SEQ ID No. 2], the KYE1 gene (L37452) from Kluyveromyces lactis NRRL Y-1140 [SEQ ID No. 3] encoding of the old yellow enzyme KYE1 of K. lactis NRRL Y-1140 [SEQ ID No. 4], the xenB gene (AF154062) from Pseudomonas fluorescens I-C [SEQ ID No. 5] encoding the xenobiotic reductase XenB of P. fluorescens I-C [SEQ ID No. 6], the ncr gene (AF093246) from Pseudomonas syringae pv. glycinea [SEQ ID No. 7] encoding the 2-cyclohexen-1-one reductase Ncr of P. syringae pv. glycinea [SEQ ID No. 8], the nemA gene (D86931) from Escherichia coli W3110 [SEQ ID No. 9] encoding the N-ethyl maleimide reductase NemA from E. coli W3110 [SEQ ID No. 10], the OYE2 gene (L06124) from Saccharomyces cerevisiae S288C [SEQ ID No. 11] encoding old yellow enzyme OYE2 from S. cerevisiae S288C [SEQ ID No. 12], and the yqjM gene (Z99116) from Bacillus subtilis str. 168 [SEQ ID No. 13] encoding YqjM from B. subtilis str. 168 [SEQ ID No. 14] were amplified from genomic DNA of the respective micro-organisms by PCR using PCR Supermix High Fidelity (Invitrogen) according to the manufacturer's specifications with the following oligonucleotides:

TABLE 2
Primer sequence used for cloning 6, 7-DAO enone reductase genes by PCR

| gene | primer | SEQ ID No. | nucleic acid sequence (5′ - 3′) |
|---|---|---|---|
| OYE C. macedoniensis | forward | 15 | AGGAGGAATTAACCATGTCGTACATGAACTT |
| AKU4588 | | | TGACCC |
| [SEQ ID No. 1] | reverse | 16 | TTAGTACTTCTTTTCCTCTTTC |
| KYE1 K. lactis NRRL Y- | forward | 17 | AGGAGGAATTAACCATGTCGTTTATGAACTT |
| 1140 | | | TGAACCAAAGCC |
| [SEQ ID No. 3] | reverse | 18 | CTATTTCTTGTAACCCTTGGCAACAGC |
| xenB P. fluorescens I-C | forward | 19 | AGGAGGAATTAACCATGGCAACTATTTTCGA |
| [SEQ ID No. 5] | | | TCCGATCAAACTGG |
| | reverse | 20 | TTACAGCGTCGGGTAGTCGATGTAGCCGACC |
| ncr P. syringae pv. | forward | 21 | AGGAGGAATTAACCATGCCGACTCTTTTCGA |
| glycinea | | | CCCC |
| [SEQ ID No. 7] | reverse | 22 | CTACTTTTGGTCAGCGGTGGG |
| nemA E. coli W3110 | forward | 23 | AGGAGGAATTAACCATGTCATCTGAAAAA |
| [SEQ ID No. 9] | | | CTGTATTCCCC |
| | reverse | 24 | TTACAACGTCGGGTAATCGGTATAGC |
| OYE2 S. cerevisiae | forward | 25 | AGGAGGAATTAACCATGCCATTTGTTAAGGA |
| S288C | | | CTTTAAGCC |
| [SEQ ID No. 11] | reverse | 26 | TTAATTTTTGTCCCAACCGAGTTTTAGAGC |
| yqjM B. subtilis str. 168 | forward | 27 | AGGAGGAATTAACCATGGCCAGAAAATTATT |
| [SEQ ID No. 13] | | | TACACC |
| | reverse | 28 | TTACCAGCCTCTTTCGTATTGAAC |

Construction of Expression Plasmids

PCR reactions were analysed by agarose gel electrophoresis and PCR products of the correct size were eluted from the gel using the QIAquick PCR purification kit (QIAGEN, Hilden, Germany). Purified PCR products were cloned into pBAD/Myc-His-DEST expression vectors using the Gateway technology (Invitrogen) via the introduced attB sites and pDONR201 (Invitrogen) as entry vector as described in the manufacturer's protocols (www.invitrogen.com). This way the expression vectors pBAD-ER_Cma harbouring [SEQ ID. No. 1], pBAD-ER_Kla harbouring [SEQ ID. No. 3], pBAD-ER_Pfl harbouring [SEQ ID. No. 5], pBAD-ER_Psy harbouring [SEQ ID. No. 7], pBAD-ER_Eco harbouring [SEQ ID. No. 9], pBAD-ER_Sce harbouring [SEQ ID. No. 11], and pBAD-ER_Bsu harbouring [SEQ ID. No. 13] were obtained, respectively. The sequences of the cloned genes were verified by DNA sequencing. The corresponding expression strains were obtained by transformation of chemically competent E. coli TOP10 (Invitrogen) with the respective pBAD-expression vectors.

Gene Synthesis and Construction of Plasmids

Synthetic genes were obtained from DNA2.0 and codon optimised for expression in E. coli according to standard procedures of DNA2.0. The codon optimised OYE gene from Candida macedoniensis AKU4588 [SEQ ID No. 1], KYE1 gene from Kluyveromyces lactis NRRL Y-1140 [SEQ ID No. 3], xenB gene from Pseudomonas fluorescens I-C [SEQ ID No. 5], ncr gene from Pseudomonas syringae pv. glycinea [SEQ ID No. 7], respectively, were codon optimised and the resulting sequences [SEQ ID No. 35, 36, 37, 38] were obtained by DNA synthesis. The gene constructs were cloned into pBAD/Myc-His-DEST expression vectors using the Gateway technology (Invitrogen) via the introduced attB sites and pDONR entry vectors (Invitrogen) as described in the manufacturer's protocols (www.invitrogen.com). This way the expression vectors pBAD-ER-co_Cma harbouring [SEQ ID No. 35], pBAD-ER-co_Kla harbouring [SEQ ID No. 36], pBAD-ER-co_Pfl harbouring [SEQ ID No. 37], and pBAD-ER-co_Psy harbouring [SEQ ID No. 38] were obtained, respectively. The corresponding expression strains were obtained by transformation of chemically competent E. coli TOP10 (Invitrogen) with the respective pBAD-expression vectors.

Cultivation of E. coli for 6,7-DAO Enone Reductase Protein Expression

Cultivations were carried out in 96-deep-well plates with 940 μl media containing 0.02% (w/v) L-arabinose. Inoculation was performed by transferring cells from frozen stock cultures with a 96-well stamp (Kühner, Birsfelden, Switzerland). Plates were incubated on an orbital shaker (Kühner; 300 rpm, 5 cm amplitude) at 25° C. for 48 h. Typically cell densities of OD₆₂₀=of 2-4 were reached.

Preparation of Cell Lysates of 6,7-DAO Enone Reductases

Cells from small scale cultivations were harvested by centrifugation and the supernatant was discarded. The cell pellets formed during centrifugation were frozen at −20° C. for at least 16 h and then thawed on ice. 500 μl of freshly prepared lysis buffer were added to each well and cells were resuspended by vigorously vortexing the plate for 2-5 min. To achieve lysis, the plate was incubated at room temperature for 30 min. To remove cell debris, the plate was centrifuged at 4° C. and 6000 g for 20 min. The supernatant was transferred to a fresh plate and kept on ice until further use.

The lysis buffer contained the ingredients, as shown in the following table:

TABLE 3

| | | |
|---|---|---|
| 1M MOPS pH 7.5 | 5 | ml |
| DNAse I grade II (Roche) | 10 | mg |
| Lysozyme (Sigma) | 200 | mg |
| MgSO₄•7H₂O | 123.2 | mg |
| dithiothreitol (DTT) | 154.2 | mg |
| H₂O (MilliQ) | Balance to 100 | ml |

The solution was freshly prepared directly before use.

2.3a Biocatalytic Synthesis of Caprolactam from 6,7-DAO

A reaction mixture was prepared comprising 20 mM 6,7-DAO, 30 mM glucose, 1 mM NADPH and 10 μml D-glucose dehydrogenase from Bacillus megaterium (catalogue no. 22.10; Julich Chiral Solutions, Jülich, Germany) in 50 mM potassium phosphate buffer, pH 7.2. To start the reaction, 400 μl of the cell lysate was added to the reaction mixture to a total volume of 550 μl. Reaction mixtures were incubated on a shaker at 28° C. for 48 h. Furthermore, a chemical blank mixture (without cell free extract) and a biological blank (E. coli TOP10 with pBAD/Myc-His C) were incubated under the same conditions. Samples were analysed by HPLC-MS. The results are summarised in the following table.

TABLE 4
Caprolactam formation from 6,7-DAO in the presence of
cell lysates from various 6,7-DAO enone reductases:

| Biocatalyst | Caprolactam concentration [mg/kg] |
|---|---|
| E. coli TOP10 pBAD-ER-co_Cma | 0.96 |
| E. coli TOP10 pBAD-ER-co_Psy | 0.59 |
| E. coli TOP10 pBAD-ER-co_Kla | 0.31 |
| E. coli TOP10 pBAD-ER_Eco | 0.31 |
| E. coli TOP10 pBAD-ER-co_Pfl | 0.27 |
| E. coli TOP10 pBAD-ER_Sce | 0.09 |
| E. coli TOP10 pBAD-ER_Bsu | 0.08 |
| E. coli TOP10 pBAD/Myc-His C | 0 |
| (biological blank) | |
| None (chemical blank) | 0 |

It is shown that the formation of caprolactam from 6,7-DAO is catalysed by the biocatalyst.

### 2.3b Bioconversion of 6,7-DAO to Caprolactam

A reaction mixture was prepared comprising components to 1 ml enzyme solution of E. coli TOP10 pBAD-ER-co_Cma (prepared as described above), 1200 U D-glucose dehydrogenase from Bacillus megaterium (catalogue no. 22.10; Julich Chiral Solutions, Jülich, Germany), 9.1 mM 6,7-DAO (containing an impurity of 3.5% (w/w) caprolactam), 0.9 mM NADPH, and 100 mM glucose. The total mixture volume was 1.1 ml.

After incubation at 37° C. for 24 h samples were taken for HPLC-UV-MS analysis. Furthermore, a chemical blank mixture (without cell free extract) and a biological blank mixture were incubated under the same conditions and sampled after the same incubation time. Biological blank cells were used as a negative control for detecting any background enzyme activity present in the host E. coli TOP10. For these reasons, the host E. coli TOP10 has been transformed with an empty pBAD vector (pBAD/Myc-His C).

The results were as follows: In the reaction mixture, 186 mg/kg caprolactam was detected. Background caprolactam concentrations in chemical blanks averaged 71.5 mg/kg (due to a caprolactam impurity present in the 6,7-DAO). In the biological blanks, no additional caprolactam was formed. From this it can be concluded than 114 mg/kg caprolactam had been converted biocatalytically from 6,7-DAO.

1. Method for preparing ε-caprolactam comprising reducing the carbon-carbon double bond of (Z)-6,7-dihydro-1H-azepin-2(5H)-one, wherein the reduction is catalysed by a biocatalyst.

2. Method according to claim 1, wherein the biocatalyst has (Z)-6,7-dihydro-1H-azepin-2(5H)-one enone reductase activity.

3. Method according to claim 2, wherein the biocatalyst comprises an enzyme selected from the group of oxidoreductases (EC1).

4. Method according to claim 3, wherein the oxidoreductase is selected from the group of oxidoreductases acting on CH—CH donors (EC1.3) and oxidoreductase acting on NADH or NADPH (EC 1.6).

5. Method according to claim 4, wherein the oxidoreductase is selected from the group of 2-enone reductases (EC 1.3.1.33) and old yellow enzymes (EC 1.6.99.1).

6. Method according to claim 1, wherein a cofactor for the enzyme is present, in particular a cofactor selected from the group of NADPH, NADH, FADH and quinones.

7. Method according to claim 1, wherein the enzyme is selected from the group of enzymes capable of catalysing (Z)-6,7-dihydro-1H-azepin-2(5H)-one enone reduction from an organism or part of an organism selected from the group of Candida, Kluyveromyces, Saccharomyces, Pseudomonas, Escherichia and Bacillus.

8. Method according to claim 1, wherein the biocatalyst comprises a polypeptide comprising an amino acid sequence represented by Sequence ID 2, 4, 6, 8, 10, 12, 14 or a homologue thereof.

9. Method according to claim 8, wherein said amino acid sequence has a sequence identity with any of said Sequence ID's of at least 80%, in particular of at least 90%, more in particular of at least 95%.

10. Method according to claim 1, wherein the method is carried out in an aqueous environment.

11. Method according to claim 1, wherein the (Z)-6,7-dihydro-1H-azepin-2(5H)-one has been prepared by removing the α-amino group from α-amino-ε-caprolactam.

12. Method according to claim 1, wherein the α-amino-ε-caprolactam has been prepared from lysine.

13. A recombinant host cell comprising a nucleic acid sequence encoding a biocatalyst with (Z)-6,7-dihydro-1H-azepin-2(5H)-one enone reductase activity.

14. A host cell according to claim 13, wherein said biocatalyst having enone reductase activity comprises a nucleic acid sequence as defined in any of Sequence ID 35-38 or a non-wild type functional analogue thereof.

15. A host cell according to claim 13, comprising a nucleic acid sequence encoding a biocatalyst with L-lysine cyclase activity.

16. Host cell according to claim 13, wherein the host cell is selected from the group of genera consisting of Aspergillus, Penicillium, Saccharomyces, Kluyveromyces, Pichia, Candida, Hansenula, Bacillus, Corynebacterium and Escherichia.

17. Polynucleotide comprising a nucleic acid sequence as defined in, any of Sequence ID 35-38 or a non-wild type functional analogue thereof.
